(19) United States
(12) Patent Application Publication
(10) Pub. No.: US 2009/0203753 A1
(43) Pub. Date: Aug. 13, 2009

(54) 7-(2-amino-1-hydroxy-ethyl)-4-hydroxybenzothiazol-2(3H)-one-derivatives as beta2 adrenoreceptor agonists

(75) Inventors: Andrew Bailey, Leicestershire (GB); Roger Bonnert, Leicestershire (GB); Alice Flaherty, Leicestershire (GB); Garry Pairaudeau, Leicestershire (GB); Michael Stocks, Leicestershire (GB)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 12/065,165

(22) PCT Filed: Aug. 28, 2006
(86) PCT No.: PCT/SE06/00980

§ 371 Date: Jul. 29, 2008

(30) Foreign Application Priority Data
Aug. 29, 2005 (SE) 0501906-2

(51) Int. Cl. A61K 31/428; C07D 277/68; A61P 11/06

(52) U.S. Cl. 514/367; 548/171

(57) ABSTRACT

The present invention provides compounds of formula (I) wherein e, R¹, R², R³, R⁴, R⁵, R^{4′}, R^{5′}, R6, R⁷, A, D, m and n are as defined in the specification, processes for their preparation, pharmaceutical compositions containing them and their use in therapy.

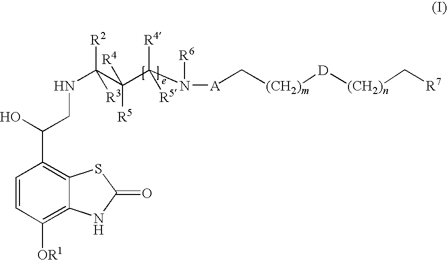

The present invention relates to benzothiazolone derivatives, processes for their preparation, pharmaceutical compositions containing them and their use in therapy.

Adreneoceptors are a group of G-protein coupled receptors divided into two major sub-families, α and β. These sub-families are further divided into sub-types of which the β sub-family has at least 3 members: β1, β2 and β3. β2 adrenoceptors (henceforth referred to as β2 receptors) are mainly expressed on smooth muscle cells.

Agonism of the β2 receptor on airway smooth muscle produces relaxation and therefore bronchodilatation. Through this mechanism, β2 agonists act as functional antagonists to all bronchoconstrictor substances such as the naturally-occurring histamine and acetylcholine as well as the experimental substances methacholine and carbachol. β2 agonists are widely used to treat airways diseases including asthma and chronic obstructive pulmonary disease (COPD), and this has been extensively reviewed in the literature and incorporated into national guidelines for the treatment of these diseases (British Guideline on the Management of Asthma, NICE guideline No. 12 on the Management of COPD). β2 agonists are classed either as short-acting or long-acting. Short-acting β2 agonists (SABAs) such as salbutamol have a duration of action of 2-4 hours. They are suitable for rescue medication during a period of acute bronchoconstriction but are not suitable for continuous medication because the beneficial effect of these drugs wears off during the night. Long-acting β2 agonists (LABAs) currently have a duration of action of about 12 hours and are administered twice daily to provide continuous bronchodilatation. They are particularly effective when administered in combination with inhaled corticosteroids. This benefit is not seen when inhaled corticosteroids are combined with SABAs (Kips and Pauwels, Am. J. Respir. Crit. Care Med., 2001, 164, 923-932). LABAs are recommended as add-on therapy to patients already receiving inhaled corticosteroids for asthma to reduce nocturnal awakening and reduce the incidence of exacerbations of the disease.

Corticosteroids and LABAs are conveniently co-administered in a single inhaler to improve patient compliance.

There are shortcomings to existing LABAs and there is a need for a new drug in this class. Salmeterol, a commonly used LABA, has a narrow safety margin and side effects related to systemic agonism of β2 receptors (such as tremor, hypokalaemia, tachycardia and hypertension) are common. Salmeterol also has a long onset of action which precludes its use as both a rescue and a maintenance therapy. All current LABAs are administered twice daily and there is a medical need for once daily treatments to improve treatment and patient compliance. Such once daily compounds, co-administered with corticosteroids, will become the mainstay of asthma treatment (Barnes, Nature Reviews, 2004, 3, 831-844). The advantages of once-daily bronchodilator treatment in COPD has been demonstrated with tiotropium, a non-selective muscarinic antagonist (Koumis and Samuel, Clin. Ther. 2005, 27(4, 377-92). There is, however, a need for a once-daily LABA for the treatment of COPD to avoid the side effects of anti-muscarinics such as tiotropium.

Benzothiazolone derivatives having dual β2 receptor and dopamine (D2) receptor agonist properties are known from WO 92/08708, WO 93/23385 and WO 97/10227. Agonists of the β2 receptor receptor are also disclosed in WO 2004/016601, WO 2004/01578 and WO 2005/074924.

In accordance with the present invention, there is therefore provided a compound of formula (I):

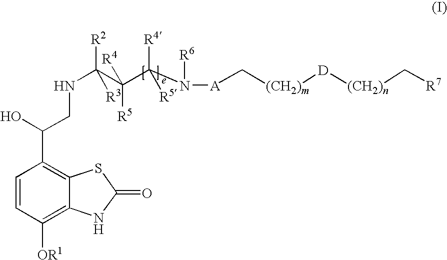

wherein
R¹ represents hydrogen or benzyl;
each of R², R³, R⁴, R⁵, R^{4′} and R^{5′} independently represents hydrogen or C₁-C₆ alkyl;
e is 0 or 1;
A represents CH₂, C(O) or S(O)₂;
D represents oxygen, sulphur or NR⁸;
m is an integer from 0 to 3;
n is an integer from 0 to 3;
R⁶ represents a group —(X)ₚ—Y-(Z)_{q}-R¹⁰;
X and Z each independently represent a C₁-C₆ alkylene group optionally substituted by halogen, trifluoromethyl, amino (NH₂), (di)-C₁-C₆ alkylamino, (di)-C₁-C₆ alkylaminocarbonyl, C₁-C₆ alkylcarbonylamino, sulphonamido (—SO₂NH₂) or (di)-C₁-C₆ alkylaminosulphonyl;
p and q each independently represent 0 or 1;
Y represents a bond, oxygen, sulphur, CH₂, C(O) or NR⁹;
R⁸ represents hydrogen or C₁-C₆ alkyl;
R⁹ represents hydrogen or C₁-C₆ alkyl;
R¹⁰ represents hydrogen, or a saturated or unsaturated 3- to 10-membered ring system comprising none, one or more heteroatoms selected from nitrogen, oxygen and sulphur, the ring system being optionally substituted by halogen, trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₆ alkyl, C₁-C₆ alkoxy, C₁-C₆ alkylcarbonyl, C₁-C₆ alkoxycarbonyl or a saturated or unsaturated 4- to 7-membered monocyclic ring system comprising none, one or more ring heteroatoms selected from nitrogen, oxygen and sulphur, the monocyclic ring system itself being optionally substituted by halogen, trifluoromethyl, hydroxyl, —NR²¹S(O)ₜR²², —NHC(O)R²³ or C₁-C₆ alkoxy;
R¹⁶, R¹⁸, R¹⁹, R²⁰, R²¹ and R²³ each independently represent hydrogen or C₁-C₆ alkyl; R¹⁵, R¹⁷ and R²² are, independently, C₁-C₆ alkyl;
r, s and t each independently represent 0, 1 or 2 (for example 2);
R⁷ represents a 5- to 14-membered aromatic or heteroaromatic ring system optionally substituted by halogen, trifluoromethyl, hydroxyl, carboxyl, C₁-C₆ alkyl (optionally substituted by —NR²⁴R²⁵), C₁-C₆ alkoxy (optionally substituted by —NR²⁶R²⁷), C₁-C₆ alkoxycarbonyl, —NR²⁸R²⁹, C₁-C₆ alkylcarbonylamino, C₁-C₆ alkylsulphonylamino, phenylsulphonylamino, —C(O)NHR³⁰, —SO₂NHR³³, C₀-C₆ alkyl-R³⁴, or a phenyl or 5- to 6-membered heteroaromatic ring (each of which is optionally substituted by halogen, trifluoromethyl, hydroxyl, C₁-C₆ alkyl, C₁-C₆ alkoxy or —NR³⁵R³⁶);
R²⁴, R²⁵, R²⁶, R²⁷, R²⁸ and R²⁹ each independently represent hydrogen or C₁-C₆ alkyl;
R³⁰ represents hydrogen, C₁-C₆ alkyl, phenyl-C₀-C₆ alkyl or C₂-C₆ alkylene-NR³¹R³²;
either R³¹ and R³² each independently represent hydrogen or C₁-C₆ alkyl, or R³¹ and R³² together with the nitrogen atom to which they are attached form a 4- to 6-membered saturated heterocyclic ring optionally comprising a further ring heteroatom selected from nitrogen and oxygen;
R³³ represents hydrogen, C₁-C₆ alkyl, phenyl-C₀-C₆ alkyl or C₂-C₆ alkylene-NR³⁷R³⁸;
R³⁴ represents a saturated, 5- or 6-membered nitrogen-containing ring;
R³⁵ and R³⁶ each independently represent hydrogen or C₁-C₆ alkyl; and
either R³⁷ and R³⁸ each independently represent hydrogen or C₁-C₆ alkyl, or R³⁷ and
R³⁸ together with the nitrogen atom to which they are attached form a 4- to 6-membered saturated heterocyclic ring optionally comprising a further ring heteroatom selected from nitrogen and oxygen;
   or a pharmaceutically acceptable salt thereof.

In the context of the present specification, unless otherwise stated, an alkyl substituent group or an alkyl moiety in a substituent group may be linear or branched. Examples of C₁-C₆ alkyl groups/moieties include methyl, ethyl, n-propyl, isopropyl, n-butyl, isobutyl, tert-butyl, n-pentyl and n-hexyl. Similarly, an alkylene group or an alkylene moiety in a substituent group may be linear or branched. Examples of C₁-C₆ alkylene groups/moieties include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene. The alkyl moieties in a di-C₁-C₆ alkylamino, di-C₁-C₆ alkylaminocarbonyl or di-C₁-C₆ alkylaminosulphonyl substituent group may be the same or different. In the definition of R¹⁰, the saturated or unsaturated 3- to 10-membered ring system and the saturated or unsaturated 4- to 7-membered monocyclic ring system may each have alicyclic or aromatic properties. An unsaturated ring system will be partially or fully unsaturated. When R³¹ and R³² (or R³⁷ and R³⁸) together represent a 4- to 6-membered saturated heterocyclic ring, it should be understood that the ring will contain no more than two ring heteroatoms: the nitrogen ring atom to which R³¹ and R³² (or R³⁷ and R³⁸) are attached and optionally a nitrogen or oxygen ring atom.

The compounds of the invention are selective β2 receptor agonists and possess properties that make them more suitable for once-a-day administration. Compounds have been optimised to have appropriate duration in an in vitro guinea pig trachea model, or mammalian model such as a histamine-challenged guinea pig. The compounds also have advantageous pharmacokinetic half lives in mammalian systems. In particular, the compounds of the invention are at least 10-fold more potent at the β2 receptor compared to the α1, β1, or dopamine (D2) receptors. The compounds are also considered to have a fast onset of action that is the time interval between administration of a compound of the invention to a patient and the compound providing symptomatic relief. Onset can be predicted in vitro using isolated trachea from guinea pig or human.

For the avoidance of doubt, when a group is described as ‘optionally substituted’ or ‘may be optionally substituted’, and whether such phrase is followed by a numerical qualification or not, said group can be unsubstituted or it can be substituted. When there is more than one substituent in the list of recited substituents and said group is substituted, said group can be substituted by the same or different substituents.

In one aspect the present invention provides a compound of formula (I), wherein: R¹ represents hydrogen or benzyl; each of R², R³, R⁴, R⁵, R^{4′} and R^{5′} independently represents hydrogen or C₁-C₆ alkyl; e is 0 or 1; A represents CH₂, C(O) or S(O)₂; D represents oxygen, sulphur or NR⁸; m is an integer from 0 to 3; n is an integer from 0 to 3; R⁶ represents a group —(X)ₚ—Y-(Z)_{q}-R¹⁰; X and Z each independently represent a C₁-C₆ alkylene group optionally substituted with at least one substituent selected from halogen, trifluoromethyl, amino (NH₂), (di)-C₁-C₆ alkylamino, (di)-C₁-C₆ alkylaminocarbonyl, C₁-C₆ alkylcarbonylamino, sulphonamido (—SO₂NH₂) and (di)-C₁-C₆ alkylaminosulphonyl; p and q each independently represent 0 or 1; Y represents a bond, oxygen, sulphur, CH₂, C(O) or NR⁹; R⁸ represents hydrogen or C₁-C₆ alkyl; R⁹ represents hydrogen or C₁-C₆ alkyl; R¹⁰ represents hydrogen, or a saturated or unsaturated 3- to 10-membered ring system optionally comprising at least one ring heteroatom selected from nitrogen, oxygen and sulphur, the ring system being optionally substituted by one or more substituents independently selected from halogen, trifluoromethyl, cyano, carboxyl, hydroxyl, nitro, S(O)ᵣR¹⁵, —NR¹⁶S(O)ₛR¹⁷, —C(O)NR¹⁵R¹⁹, —NHC(O)R²⁰, C₁-C₆ alkyl, C₁-C₆ alkoxy, C₁-C₆ alkylcarbonyl, C₁-C₆ alkoxycarbonyl and a saturated or unsaturated 4- to 7-membered monocyclic ring system optionally comprising at least one ring heteroatom selected from nitrogen, oxygen and sulphur, the monocyclic ring system itself being optionally substituted by at least one substituent selected from halogen, trifluoromethyl, hydroxyl, —NR²¹S(O)ₜR²², —NHC(O)R²³ and C₁-C₆ alkoxy; R¹⁵, R¹⁶, R¹⁷, R¹⁸, R¹⁹, R²⁰, R²¹, R²² and R²³ each independently represent hydrogen or C₁-C₆ alkyl; r, s and t each independently represent 0, 1 or 2; R⁷ represents a 6- to 14-membered aromatic or heteroaromatic ring system optionally substituted by one or more substituents independently selected from halogen, trifluoromethyl, hydroxyl, carboxyl, C₁-C₆ alkyl (optionally substituted by at least one —NR²⁴R²⁵), C₁-C₆ alkoxy (optionally substituted by at least one —NR²⁶R²⁷), C₁-C₆ alkoxycarbonyl, —NR²⁸R²⁹, C₁-C₆ alkylcarbonylamino, C₁-C₆ alkylsulphonylamino, phenylsulphonylamino, —C(O)NHR³⁰, —SO₂NHR³³, C₀-C₆ alkyl-R³⁴, and a phenyl or 5- to 6-membered heteroaromatic ring (each of which may be optionally substituted by one or more substituents independently selected from halogen, trifluoromethyl, hydroxyl, C₁-C₆ alkyl, C₁-C₆ alkoxy and —NR³⁵R³⁶); R²⁴, R²⁵, R²⁶, R²⁷, R²⁸ and R²⁹ each independently represent hydrogen or C₁-C₆ alkyl; R³⁰ represents hydrogen, C₁-C₆ alkyl, phenyl-C₀-C₆ alkyl or C₂-C₆ alkylene-NR³¹R³²; either R³¹ and R³² each independently represent hydrogen or C₁-C₆ alkyl, or R³¹ and R³² together with the nitrogen atom to which they are attached form a 4- to 6-membered saturated heterocyclic ring optionally comprising a further ring heteroatom selected from nitrogen and oxygen; R³³ represents hydrogen, C₁-C₆ alkyl, phenyl-C₀-C₆ alkyl or C₂-C₆ alkylene-NR³⁷R³⁸; R³⁴ represents a saturated, 5- or 6-membered nitrogen-containing ring; R³⁵ and R³⁶ each independently represent hydrogen or C₁-C₆ alkyl; and either R³⁷ and R³⁸ each independently represent hydrogen or C₁-C₆ alkyl, or R³⁷ and R³⁸ together with the nitrogen atom to which they are attached form a 4- to 6-membered saturated heterocyclic ring optionally comprising a further ring heteroatom selected from nitrogen and oxygen; or a pharmaceutically acceptable salt thereof.

In an embodiment of the invention R¹ is hydrogen.

In another embodiment of the invention R¹ is benzyl.

In an embodiment of the invention, each of R², R³, R⁴, R⁵ and, if present, R^{4′} and R^{5′} independently represents hydrogen or C₁-C₆, or C₁-C₄, or C₁-C₂ alkyl.

In another embodiment, each of R², R³, R⁴, R⁵ and, if present, R^{4′} and R^{5′} represents hydrogen.

In a further embodiment of the invention e is 0.

In a still further embodiment of the invention A represents C(O) or CH₂.

In an embodiment of the invention, A represents C(O).

In another embodiment of the invention, A represents CH₂.

In an embodiment of the invention, D represents oxygen.

In an embodiment of the invention, m is an integer 0, 1 2 or 3, for example, 1.

In an embodiment of the invention, n is an integer 0, 1 2 or 3, for example, 1.

In a still further embodiment of the invention X and Z each independently represent a C₁-C₆, or C₁-C₄, or C₁-C₂ alkylene group optionally substituted by (for example with none, one, two or three substituents) halogen (e.g. fluorine, chlorine, bromine or iodine), trifluoromethyl, amino, (di)-C₁-C₆, or C₁-C₄, or C₁-C₂ alkylamino (e.g. methylamino, ethylamino, dimethylamino or diethylamino), (di)-C₁-C₆, or C₁-C₄, or C₁-C₂ alkylaminocarbonyl (e.g. methylaminocarbonyl, ethylaminocarbonyl, dimethylaminocarbonyl or diethylaminocarbonyl), C₁-C₆, or C₁-C₄, or C₁-C₂ alkylcarbonylamino (e.g. methylcarbonylamino or ethylcarbonylamino), sulphonamido or (di)-C₁-C₆, or C₁-C₄, or C₁-C₂ alkylaminosulphonyl (e.g. methylaminosulphonyl, ethylaminosulphonyl, dimethylaminsulphonyl or diethylaminsulphonyl).

Thus the invention provides a compound of formula (I) wherein X and Z each independently represent a C₁-C₆, or C₁-C₄, or C₁-C₂ alkylene group optionally substituted with at least one substituent (e.g. one, two or three substituents independently) selected from halogen (e.g. fluorine, chlorine, bromine or iodine), trifluoromethyl, amino, (di)-C₁-C₆, or C₁-C₄, or C₁-C₂ alkylamino (e.g. methylamino, ethylamino, dimethylamino or diethylamino), (di)-C₁-C₆, or C₁-C₄, or C₁-C₂ alkylaminocarbonyl (e.g. methylaminocarbonyl, ethylaminocarbonyl, dimethylaminocarbonyl or diethylaminocarbonyl), C₁-C₆, or C₁-C₄, or C₁-C₂ alkylcarbonylamino (e.g. methylcarbonylamino or ethylcarbonylamino), sulphonamido and (di)-C₁-C₆, or C₁-C₄, or C₁-C₂ alkylaminosulphonyl (e.g. methylaminosulphonyl, ethylaminosulphonyl, dimethylaminsulphonyl or diethylaminsulphonyl).

In one embodiment, X represents a C₁-C₄ alkylene group.

In another embodiment, Z represents a C₁-C₄ alkylene group.

In an embodiment of the invention, p is 0 and q is 1.

In another embodiment, p is 1 and q is 0.

In still another embodiment, p and q are either both 0 or 1.

In an embodiment of the invention Y represents a bond, CH₂ or NR⁹.

In an embodiment of the invention R⁸ represents hydrogen or C₁-C₆, or C₁-C₄, or C₁-C₂ alkyl.

In an embodiment of the invention R⁹ represents hydrogen or C₁-C₆, or C₁-C₄, or C₁-C₂ alkyl.

In a further embodiment the present invention provides a compound of formula (I) wherein p and q each independently represent 0 or 1; X and Z each independently represent an unsubstituted C₁-C₆ alkylene group and Y represents a bond, CH₂ or NR⁹. R⁹ is, for example, hydrogen or C₁-C₄ alkyl (such as methyl or ethyl).

In another embodiment of the invention R¹⁰ represents hydrogen, or a saturated or unsaturated 3- to 10-membered (e.g. 3-, 4- or 5- to 6-, 7-, 8-, 9- or 10-membered) ring system optionally comprising none, one or more ring heteroatoms (e.g. none, one, two, three or four ring heteroatoms) independently selected from nitrogen, oxygen and sulphur, the ring system being optionally substituted by (e.g. by none, one, two, three or four substituents) halogen (e.g. fluorine, chlorine, bromine or iodine), trifluoromethyl, cyano, carboxyl, hydroxyl, nitro, —S(O)ᵣR¹⁵, —NR¹⁶S(O)ₛR¹⁷, —C(O)NR¹⁵R¹⁹, —NHC(O)R²⁰, C₁-C₆, or C₁-C₄, or C₁-C₂ alkyl, C₁-C₆, or C₁-C₄, or C₁-C₂ alkoxy, C₁-C₆, or C₁-C₄, or C₁-C₂ alkylcarbonyl, C₁-C₆, or C₁-C₄, or C₁-C₂ alkoxycarbonyl or a saturated or unsaturated 4-, 5-, 6- or 7-membered monocyclic ring system optionally comprising none, one or more ring heteroatoms (e.g. none, one, two, three or four ring heteroatoms) independently selected from nitrogen, oxygen and sulphur, the monocyclic ring system itself being optionally substituted by (e.g. none, one, two, three or four substituents) halogen (e.g. fluorine, chlorine, bromine or iodine), trifluoromethyl, hydroxyl, —NR²¹S(O)ₜR²², —NHC(O)R²³ or C₁-C₆, or C₁-C₄, or C₁-C₂ alkoxy.

Thus the invention provides a compound of formula (I) wherein R¹⁰ represents hydrogen, or a saturated or unsaturated 3- to 10-membered (e.g. 3-, 4- or 5- to 6-, 7-, 8-, 9- or 10-membered) ring system optionally comprising at least one ring heteroatom (e.g. one, two, three or four ring heteroatoms independently) selected from nitrogen, oxygen and sulphur, the ring system being optionally substituted by one or more (e.g. one, two, three or four) substituents independently selected from halogen (e.g. fluorine, chlorine, bromine or iodine), trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₆, or C₁-C₄, or C₁-C₂ alkyl, C₁-C₆, or C₁-C₄, or C₁-C₂ alkoxy, C₁-C₆, or C₁-C₄, or C₁-C₂ alkylcarbonyl, C₁-C₆, or C₁-C₄, or C₁-C₂ alkoxycarbonyl and a saturated or unsaturated 4-, 5-, 6- or 7-membered monocyclic ring system optionally comprising at least one ring heteroatom (e.g. one, two, three or four ring heteroatoms independently) selected from nitrogen, oxygen and sulphur, the monocyclic ring system itself being optionally substituted by at least one substituent (e.g. one, two, three or four substituents independently) selected from halogen (e.g. fluorine, chlorine, bromine or iodine), trifluoromethyl, hydroxyl, —NR²¹S(O)ₜR²², —NHC(O)R²³ and C₁-C₆, or C₁-C₄, or C₁-C₂ alkoxy.

Examples of saturated or unsaturated 3- to 10-membered ring systems that may be used, which may be monocyclic or polycyclic (e.g. bicyclic) in which the two or more rings are fused, include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, diazabicyclo[2.2.1]hept-2-yl, naphthyl, benzofuranyl, benzothienyl, benzodioxolyl, quinolinyl, oxazolyl, 2,3-dihydrobenzofuranyl, tetrahydropyranyl, pyrazolyl, pyrazinyl, thiazolidinyl, indanyl, thienyl, isoxazolyl, pyridazinyl, thiadiazolyl, pyrrolyl, furanyl, thiazolyl, indolyl, imidazolyl, pyrimidinyl, benzimidazolyl, triazolyl, tetrazolyl and pyridinyl. For example the saturated or unsaturated 3- to 10-membered ring system is phenyl.

Examples of saturated or unsaturated 4- to 7-membered monocyclic ring systems that may be used include cyclobutyl, cyclopentyl, cyclohexyl, pyrrolidinyl, piperazinyl, morpholinyl, furanyl, thienyl, pyrrolyl, phenyl, oxazolyl, 1,2,4-oxadiazolyl, 1,3,4-oxadiazolyl, isoxazolyl, imidazolyl, pyrazolyl, thiazolyl, triazolyl, thiadiazolyl, pyridinyl, pyrimidinyl, pyrazinyl and tetrazolyl.

In an embodiment of the invention, R¹⁰ represents hydrogen, or a saturated or unsaturated 5- or 6-membered ring system optionally comprising none, one or more ring heteroatoms (e.g. none, one or two ring heteroatoms) independently selected from nitrogen and oxygen, the ring system being optionally substituted (e.g. none, one, two, three or four substituents) by halogen (e.g. fluorine, chlorine, bromine or iodine), trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₆, or C₁-C₄, or C₁-C₂ alkyl, C₁-C₆, or C₁-C₄, or C₁-C₂ alkoxy, C₁-C₆, or C₁-C₄, or C₁-C₂ alkylcarbonyl, C₁-C₆, or C₁-C₄, or C₁-C₂ alkoxycarbonyl or a saturated or unsaturated 4-, 5-, 6- or 7-membered monocyclic ring system optionally comprising none, one or more ring heteroatoms (e.g. none, one, two, three or four ring heteroatoms) independently selected from nitrogen, oxygen and sulphur, the monocyclic ring system itself being optionally substituted by (e.g. none, one, two, three or four substituents) halogen (e.g. fluorine, chlorine, bromine or iodine), trifluoromethyl, hydroxyl, —NR²¹S(O)ₜR²², —NHC(O)R²³ or C₁-C₆, or C₁-C₄, or C₁-C₂ alkoxy.

Thus the invention provides a compound of formula (I) wherein R¹⁰ represents hydrogen, or a saturated or unsaturated 5- or 6-membered ring system optionally comprising at least one ring heteroatom (e.g. one or two ring heteroatoms independently) selected from nitrogen and oxygen, the ring system being optionally substituted by one or more (e.g. one, two, three or four) substituents independently selected from halogen (e.g. fluorine, chlorine, bromine or iodine), trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₆, or C₁-C₄, or C₁-C₂ alkyl, C₁-C₆, or C₁-C₄, or C₁-C₂ alkoxy, C₁-C₆, or C₁-C₄, or C₁-C₂ alkylcarbonyl, C₁-C₆, or C₁-C₄, or C₁-C₂ alkoxycarbonyl and a saturated or unsaturated 4-, 5-, 6- or 7-membered monocyclic ring system optionally comprising at least one ring heteroatom (e.g. one, two, three or four ring heteroatoms independently) selected from nitrogen, oxygen and sulphur, the monocyclic ring system itself being optionally substituted by at least one substituent (e.g. one, two, three or four substituents independently) selected from halogen (e.g. fluorine, chlorine, bromine or iodine), trifluoromethyl, hydroxyl, —NR²¹S(O)ₜR²², —NHC(O)R²³ and C₁-C₆, or C₁-C₄, or C₁-C₂ alkoxy.

In another embodiment R¹⁰ represents hydrogen, or a saturated or unsaturated 5- or 6-membered ring system optionally comprising none, one or more ring heteroatoms (e.g. none, one or two ring heteroatoms) independently selected from nitrogen and oxygen, the ring system being optionally substituted (for example none, one or two substituents) independently selected from halogen (e.g. fluorine, chlorine, bromine or iodine), trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₄ or C₁-C₂ alkyl, C₁-C₄ or C₁-C₂ alkoxy, C₁-C₄ or C₁-C₂ alkylcarbonyl, C₁-C₄ or C₁-C₂ alkoxycarbonyl or a saturated or unsaturated 5- or 6-membered monocyclic ring system optionally comprising none, one or more ring heteroatom (e.g. none, one or two ring heteroatoms) independently selected from nitrogen, oxygen and sulphur, the monocyclic ring system itself being optionally substituted by (e.g. none, one or two substituents) halogen (e.g. fluorine, chlorine, bromine or iodine), trifluoromethyl, hydroxyl, —NR²¹S(O)ₜR²², —NHC(O)R²³ or C₁-C₄ or C₁-C₂ alkoxy.

Thus the invention provides a compound of formula (I) wherein R¹⁰ represents hydrogen, or a saturated or unsaturated 5- or 6-membered ring system optionally comprising at least one ring heteroatom (e.g. one or two ring heteroatoms independently) selected from nitrogen and oxygen, the ring system being optionally substituted by one or two substituents independently selected from halogen (e.g. fluorine, chlorine, bromine or iodine), trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₄ or C₁-C₂ alkyl, C₁-C₄ or C₁-C₂ alkoxy, C₁-C₄ or C₁-C₂ alkylcarbonyl, C₁-C₄ or C₁-C₂ alkoxycarbonyl and a saturated or unsaturated 5- or 6-membered monocyclic ring system optionally comprising at least one ring heteroatom (e.g. one or two ring heteroatoms independently) selected from nitrogen, oxygen and sulphur, the monocyclic ring system itself being optionally substituted by at least one substituent (e.g. one or two substituents independently) selected from halogen (e.g. fluorine, chlorine, bromine or iodine), trifluoromethyl, hydroxyl, —NR²¹S(O)ₜR²², —NHC(O)R²³ and C₁-C₄ or C₁-C₂ alkoxy.

In a further embodiment R¹⁰ represents hydrogen, or a saturated or unsaturated 5- or 6-membered ring system optionally comprising one or two ring heteroatoms independently selected from nitrogen and oxygen, the ring system being optionally substituted by (for example by none, one or two substituents) halogen (e.g. fluorine, chlorine, bromine or iodine), trifluoromethyl, carboxyl, hydroxyl, —S(O)ᵣR¹⁵, —NR¹⁶S(O)ₛR¹⁷, —C(O)NR¹⁸R¹⁹, —NHC(O)R²⁰, C₁-C₄ or C₁-C₂ alkyl, C₁-C₄ or C₁-C₂ alkoxy, C₁-C₄ or C₁-C₂ alkylcarbonyl or C₁-C₄ or C₁-C₂ alkoxycarbonyl.

Thus the invention provides a compound of formula (I) wherein R¹⁰ represents hydrogen, or a saturated or unsaturated 5- or 6-membered ring system optionally comprising one or two ring heteroatoms independently selected from nitrogen and oxygen, the ring system being optionally substituted by one or two substituents independently selected from halogen (e.g. fluorine, chlorine, bromine or iodine), trifluoromethyl, carboxyl, hydroxyl, —S(O)ᵣR¹⁵, —NR¹⁶S(O)ₛR¹⁷—C(O)NR¹⁸R¹⁹, —NHC(O)R²⁰, C₁-C₄ or C₁-C₂ alkyl, C₁-C₄ or C₁-C₂ alkoxy, C₁-C₄ or C₁-C₂ alkylcarbonyl and C₁-C₄ or C₁-C₂ alkoxycarbonyl. For example R¹⁵ and R¹⁷ are, independently, C₁-C₄ alkyl; R¹⁶, R¹⁸, R¹⁹ and R²⁰ are, independently, hydrogen or C₁-C₄ alkyl; and r and s are both 2.

In a further embodiment R¹⁰ represents hydrogen or phenyl.

In yet another aspect of the invention R⁶ is hydrogen, phenyl or (CH₂)ₖR^{10a}; wherein k is 1, 2, 3 or 4; R^{10a} is hydrogen, phenyl or NR^{9a}R^{9b}; and R^{9a} and R^{9b} are, independently, C₁₋₄ alkyl (for example methyl or ethyl).

In a further aspect of the invention R⁶ is (CH₂)ₖR^{10a}; wherein k is 1, 2, 3 or 4; R^{10a} is hydrogen, phenyl or NR^{9a}R^{9b}; and R^{9a} and R^{9b} are, independently, C₁₋₄ alkyl (for example methyl or ethyl).

In a still further aspect of the invention R⁶ is (CH₂)ₖR^{10a}; wherein k is 1, 2, 3 or 4 (for example 2); R^{10a} is NR^{9a}R^{9b}; and R^{9a} and R^{9b} are, independently, C₁₋₄ alkyl (for example methyl or ethyl).

In a still further embodiment R¹⁵, R¹⁶, R¹⁷, R¹⁸, R¹⁹, R²⁰, R²¹, R²² and R²³ each independently represent hydrogen or C₁-C₆, or C₁-C₄, or C₁-C₂ alkyl.

In another embodiment R⁷ represents a 5- to 14-membered (5-, 6-, 7-, 8-, 9-, 10-, 11-, 12-, 13- or 14-membered) aromatic or heteroaromatic ring system optionally substituted by (e.g. by none, one, two, three or four substituents) halogen (e.g. fluorine, chlorine, bromine or iodine), trifluoromethyl, hydroxyl, carboxyl, C₁-C₆, or C₁-C₄, or C₁-C₂ alkyl (optionally substituted by (e.g. none, one or two)-NR²⁴R²⁵), C₁-C₆, or C₁-C₄, or C₁-C₂ alkoxy (optionally substituted by (e.g. none, one or two)-NR²⁶R²⁷), C₁-C₆, or C₁-C₄, or C₁-C₂ alkoxycarbonyl, —NR²⁸R²⁹, C₁-C₆, or C₁-C₄, or C₁-C₂ alkylcarbonylamino, C₁-C₆, or C₁-C₄, or C₁-C₂ alkylsulphonylamino, phenylsulphonylamino, —C(O)NHR³⁰, —SO₂NHR³³, C₀-C₆, or C₀-C₄, or C₀-C₂ alkyl-R³⁴, or phenyl or 5- to 6-membered heteroaromatic ring (each of which is optionally substituted by (e.g. by none, one, two, three or four substituents) halogen such as fluorine, chlorine, bromine or iodine, trifluoromethyl, hydroxyl, C₁-C₆, or C₁-C₄, or C₁-C₂ alkyl, C₁-C₆, or C₁-C₄, or C₁-C₂ alkoxy or —NR³⁵R³⁶).

Thus the invention provides a compound of formula (I) wherein R⁷ represents a 6- to 14-membered (6-, 7-, 8-, 9-, 10-, 11-, 12-, 13- or 14-membered) aromatic or heteroaromatic ring system optionally substituted by one or more (e.g. one, two, three or four) substituents independently selected from halogen (e.g. fluorine, chlorine, bromine or iodine), trifluoromethyl, hydroxyl, carboxyl, C₁-C₆, or C₁-C₄, or C₁-C₂ alkyl (optionally substituted by at least one, e.g. one or two, —NR²⁴R²⁵), C₁-C₆, or C₁-C₄, or C₁-C₂ alkoxy (optionally substituted by at least one, e.g. one or two, —NR²⁶R²⁷), C₁-C₆, or C₁-C₄, or C₁-C₂ alkoxycarbonyl, —NR²⁸R²⁹, C₁-C₆, or C₁-C₄, or C₁-C₂ alkylcarbonylamino, C₁-C₆, or C₁-C₄, or C₁-C₂ alkylsulphonylamino, phenylsulphonylamino, —C(O)NHR³⁰, —SO₂NHR³³, C₀-C₆, or C₀-C₄, or C₀-C₂ alkyl-R³⁴, and phenyl or 5- to 6-membered heteroaromatic ring (each of which may be optionally substituted by one or more, e.g. one, two, three or four, substituents independently selected from halogen such as fluorine, chlorine, bromine or iodine, trifluoromethyl, hydroxyl, C₁-C₆, or C₁-C₄, or C₁-C₂ alkyl, C₁-C₆, or C₁-C₄, or C₁-C₂ alkoxy and —NR³⁵R³⁶).

When R⁷ represents an optionally substituted 5- to 14-membered (for example 6- to 14-membered) heteroaromatic ring system, the ring system comprises from 1 to 4 ring heteroatoms independently selected from nitrogen, oxygen and sulphur. Similarly, if a substituent in R⁷ represents an optionally substituted 5- to 6-membered heteroaromatic ring, the ring comprises from 1 to 4 ring heteroatoms independently selected from nitrogen, oxygen and sulphur.

Examples of 5- to 14-membered aromatic or heteroaromatic ring systems that may be used, which may be monocyclic or polycyclic (e.g. bicyclic or tricyclic) in which the two or more rings are fused, include one or more (in any combination) of phenyl, naphthyl, pyridinyl, pyridazinyl, pyrimidinyl, pyrazinyl, 1,3,5-triazinyl, 1,2,4-triazinyl, azepinyl, oxepinyl, thiepinyl, indenyl, benzofuranyl, isobenzofuranyl, benzothiophenyl, indolyl, isoindolyl, benzimidazolyl, indazolyl, benzisoxazolyl, benzoxazolyl, benzothiazolyl, quinolinyl, isoquinolinyl, quinazolinyl, quinoxalinyl and dibenzofuranyl. Preferred ring systems include phenyl and naphthyl.

Examples of 5- to 6-membered heteroaromatic rings include pyridinyl, triazolyl and tetrazolyl.

In an embodiment of the invention R⁷ represents a 5- to 10-membered aromatic or heteroaromatic ring system optionally substituted by (e.g. by none, one or two substituents) halogen (e.g. fluorine, chlorine, bromine or iodine), trifluoromethyl, hydroxyl, carboxyl, C₁-C₄ or C₁-C₂ alkyl (optionally substituted (e.g. none, one or two)-NR²⁴R²⁵), C₁-C₄ or C₁-C₂ alkoxy (optionally substituted (e.g. none, one or two)-NR²⁶R²⁷), C₁-C₄ or C₁-C₂ alkoxycarbonyl, —NR²⁸R²⁹, C₁-C₄ or C₁-C₂ alkylcarbonylamino, C₁-C₄ or C₁-C₂ alkylsulphonylamino, phenylsulphonylamino, —C(O)NHR³⁰, —SO₂NHR³³, C₀-C₄ or C₀-C₂ alkyl-R³⁴, phenyl or a 5- to 6-membered heteroaromatic ring.

Thus the invention provides a compound of formula (I) wherein R⁷ represents a 6- to 10-membered aromatic or heteroaromatic ring system optionally substituted by one or more (e.g. one or two) substituents independently selected from halogen (e.g. fluorine, chlorine, bromine or iodine), trifluoromethyl, hydroxyl, carboxyl, C₁-C₄ or C₁-C₂ alkyl (optionally substituted by at least one, e.g. one or two, —NR²⁴R²⁵), C₁-C₄, or C₁-C₂ alkoxy (optionally substituted by at least one, e.g. one or two, —NR²⁶R²⁷), C₁-C₄ or C₁-C₂ alkoxycarbonyl, —NR²⁸R²⁹, C₁-C₄ or C₁-C₂ alkylcarbonylamino, C₁-C₄ or C₁-C₂ alkylsulphonylamino, phenylsulphonylamino, —C(O)NHR³⁰, —SO₂NHR³³, C₀-C₄ or C₀-C₂ alkyl-R³⁴, phenyl and a 5- to 6-membered heteroaromatic ring.

In another embodiment R⁷ represents a 6- to 10-membered aromatic ring system optionally substituted by none, one or two substituents independently selected from halogen (e.g. fluorine, chlorine, bromine or iodine), trifluoromethyl, hydroxyl, carboxyl, C₁-C₄ or C₁-C₂ alkyl (optionally substituted by none, one or two —NR²⁴R²⁵), C₁-C₄ or C₁-C₂ alkoxy (optionally substituted by none, one or two —NR²⁶R²⁷), C₁-C₄ or C₁-C₂ alkoxycarbonyl, —NR²⁸R²⁹, C₁-C₄ or C₁-C₂ alkylcarbonylamino, C₁-C₄ or C₁-C₂ alkylsulphonylamino, phenylsulphonylamino, —C(O)NHR³⁰, —SO₂NHR³³, C₀-C₄ or C₀-C₂ alkyl-R³⁴, phenyl and a 5- to 6-membered heteroaromatic ring.

Thus the invention provides a compound of formula (I) wherein R⁷ represents a 6- to 10-membered aromatic ring system optionally substituted by one or two substituents independently selected from halogen (e.g. fluorine, chlorine, bromine or iodine), trifluoromethyl, hydroxyl, carboxyl, C₁-C₄ or C₁-C₂ alkyl (optionally substituted by at least one, e.g. one or two, —NR²⁴R²⁵), C₁-C₄ or C₁-C₂ alkoxy (optionally substituted by at least one, e.g. one or two, —NR²⁶R²⁷), C₁-C₄ or C₁-C₂ alkoxycarbonyl, —NR²⁸R²⁹, C₁-C₄ or C₁-C₂ alkylcarbonylamino, C₁-C₄ or C₁-C₂ alkylsulphonylamino, phenylsulphonylamino, —C(O)NHR³⁰, —SO₂NHR³³, C₀-C₄ or C₀-C₂ alkyl-R³⁴, phenyl and a 5- to 6-membered heteroaromatic ring.

In a further embodiment R⁷ represents an unsubstituted 6- to 10-membered aromatic ring system.

In a further embodiment R²⁴, R²⁵, R²⁶, R²⁷, R²⁸ and R²⁹ each independently represent hydrogen or C₁-C₆, or C₁-C₄, or C₁-C₂ alkyl. It should be understood that if there is more than one group —NR²⁴R²⁵, the groups may be the same as, or different from, one another. Similar comments apply if there is more than one group —NR²⁶R²⁷.

In a further embodiment R³⁰ represents hydrogen; C₁-C₆, or C₁-C₄, or C₁-C₂ alkyl; phenyl-C₀-C₆, or C₀-C₄, or C₀-C₂ alkyl (e.g. phenyl or benzyl); or C₂-C₆ or C₂-C₄ alkylene-NR³¹R³² and either R³¹ and R³² each independently represent hydrogen or C₁-C₆, or C₁-C₄, or C₁-C₂ alkyl, or R³¹ and R³² together with the nitrogen atom to which they are attached form a 4- to 6-membered saturated heterocyclic ring optionally comprising a further ring heteroatom selected from nitrogen and oxygen such as azetidinyl, pyrrolidinyl, piperidinyl, piperazinyl or morpholinyl.

In a further embodiment R³³ represents hydrogen; C₁-C₆, or C₁-C₄, or C₁-C₂ alkyl; phenyl-C₀-C₆, or C₀-C₄, or C₀-C₂ alkyl (e.g. phenyl or benzyl); or C₂-C₆ or C₂-C₄ alkylene-NR³⁷R³⁸ and either R³⁷ and R³⁸ each independently represent hydrogen or C₁-C₆, or C₁-C₄, or C₁-C₂ alkyl, or R³⁷ and R³⁸ together with the nitrogen atom to which they are attached form a 4- to 6-membered saturated heterocyclic ring optionally comprising a further ring heteroatom selected from nitrogen and oxygen such as azetidinyl, pyrrolidinyl, piperidinyl, piperazinyl or morpholinyl.

In a further embodiment R³⁴ represents a saturated, 5- or 6-membered nitrogen-containing ring, e.g. a ring containing one or two ring nitrogen atoms such as hydantoin.

In a further embodiment R³⁵ and R³⁶ each independently represent hydrogen or C₁-C₆, or C₁-C₄, or C₁-C₂ alkyl.

In an embodiment of the invention,
R¹ represents hydrogen or benzyl;
e is 0;
each of R², R³, R⁴ and R⁵ represents hydrogen;
A represents C(O) or CH₂;
D represents oxygen;
m is 1;
n is 1;
R⁶ represents a group —(X)ₚ—Y-(Z)_{q}-R¹⁰;
X represents a C₁-C₄ alkylene group;
Z represents a C₁-C₄ alkylene group;
p and q each independently represent 0 or 1;
Y represents a bond, CH₂ or NR⁹;
R⁹ represents ethyl;
R¹⁰ represents hydrogen or phenyl; and
R⁷ represents an unsubstituted 6- to 10-membered aromatic ring system.

Examples of compounds of the invention include:
N-[2-[2-(4-Benzyloxy-2-oxo-3H-benzothiazol-7-yl)-2-hydroxy-ethyl]aminoethyl]-N-butyl-3-phenethyloxy-propanamide,
N-Butyl-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide,
N-[2-({(2R)-2-[4-(Benzyloxy)-2-oxo-2,3-dihydro-1,3-benzothiazol-7-yl]-2-hydroxyethyl}amino)ethyl]-3-(2-phenylethoxy)-N-(2-phenylethyl)propanamide,
N-(2-{[(2R)-2-Hydroxy-2-(4-hydroxy-2-oxo-2,3-dihydro-1,3-benzothiazol-7-yl)ethyl]amino}ethyl)-3-(2-phenylethoxy)-N-(2-phenylethyl)propanamide,
N-(2-{[(2R)-2-Hydroxy-2-(4-hydroxy-2-oxo-2,3-dihydro-1,3-benzothiazol-7-yl)ethyl]amino}ethyl)-N-methyl-3-(2-phenylethoxy)propanamide,
N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide,
4-Hydroxy-7-{(1R)-1-hydroxy-2-[(2-{[3-(2-phenylethoxy)propyl]amino}-ethyl)amino]ethyl}-1,3-benzothiazol-2(3H)-one, or
3-[2-(3-Chlorophenyl)ethoxy]-N-[2-(diethylamino)ethyl]-N-(2-{[(2R)-2-hydroxy-2-(4-hydroxy-2-oxo-2,3-dihydro-1,3-benzothiazol-7-yl)ethyl]amino}ethyl)propanamide
   or a pharmaceutically acceptable salt of any one thereof. For example the following compounds:
N-[2-[2-(4-Benzyloxy-2-oxo-3H-benzothiazol-7-yl)-2-hydroxy-ethyl]aminoethyl]-N-butyl-3-phenethyloxy-propanamide,
N-Butyl-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide hydrochloride,
N-[2-({(2R)-2-[4-(Benzyloxy)-2-oxo-2,3-dihydro-1,3-benzothiazol-7-yl]-2-hydroxyethyl}amino)ethyl]-3-(2-phenylethoxy)-N-(2-phenylethyl)propanamide,
N-(2-{[(2R)-2-Hydroxy-2-(4-hydroxy-2-oxo-2,3-dihydro-1,3-benzothiazol-7-yl)ethyl]amino}ethyl)-3-(2-phenylethoxy)-N-(2-phenylethyl)propanamide,
N-(2-{[(2R)-2-Hydroxy-2-(4-hydroxy-2-oxo-2,3-dihydro-1,3-benzothiazol-7-yl)ethyl]amino}ethyl)-N-methyl-3-(2-phenylethoxy)propanamide trifluoroacetate,
N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide,
4-Hydroxy-7-{(1R)-1-hydroxy-2-[(2-{[3-(2-phenylethoxy)propyl]amino}-ethyl)amino]ethyl}-1,3-benzothiazol-2(3H)-one, or,
3-[2-(3-Chlorophenyl)ethoxy]-N-[2-(diethylamino)ethyl]-N-(2-{[(2R)-2-hydroxy-2-(4-hydroxy-2-oxo-2,3-dihydro-1,3-benzothiazol-7-yl)ethyl]amino}ethyl)propanamide.

The present invention further provides a process for the preparation of a compound of formula (I) or a pharmaceutically acceptable salt thereof as defined above which comprises,

(a) reacting a compound of formula (II)

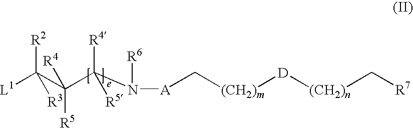

wherein L¹ represents a leaving group (e.g. chlorine, bromine, iodine, methanesulfonate or para-toluenesulfonate) and e, R², R³, R⁴, R⁵, R^{4′}, R^{5′}, R⁶, R⁷, A, D, m and n are as defined in formula (I), with a compound of formula (III) or a suitable salt thereof (e.g. hydrobromide or hydrochloride salt)

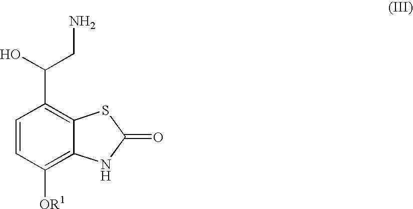

wherein R¹ is as defined in formula (I), in the presence of a base (e.g. potassium carbonate, triethylamine or diisopropylethylamine); or
(b) when R² and R³ each represent hydrogen, reacting a compound of formula (IV)

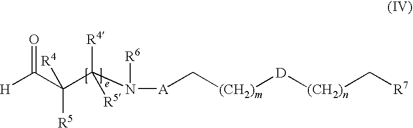

wherein e, R⁴, R⁵, R^{4′}, R^{5′}, R⁶, R⁷, A, D, m and n are as defined in formula (I), with a compound of formula (III) or a suitable salt thereof as defined in (a) above in the presence of a suitable reducing agent (e.g. sodium cyanoborohydride, sodium triacetoxyborohydride, or hydrogen in the presence of a palladium on carbon or palladium oxide catalyst); or
(c) when R² and R³ each represent hydrogen, contacting a compound of formula (V)

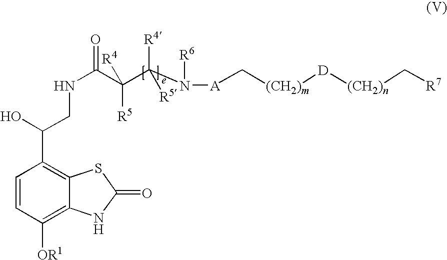

wherein e, R¹, R⁴, R⁵, R^{4′}, R^{5′}, R⁶, R⁷, A, D, m and n are as defined in formula (I) with a suitable reducing agent (e.g. lithium aluminium hydride or borane tetrahydrofuran complex);
and optionally after (a), (b) or (c) carrying out one or more of the following:
converting the compound obtained to a further compound of the invention
forming a pharmaceutically acceptable salt of the compound.

In process (a), the reaction may conveniently be carried out in an organic solvent such as N,N-dimethylformamide, ethanol, n-butanol or dimethyl sulfoxide, at a temperature, for example, in the range from 50 to 140° C.

In process (b), the reaction may conveniently be carried out in an organic solvent such as methanol, ethanol, dichloromethane, acetic acid, or N,N-dimethylformamide containing up to 10% w of water and acetic acid.

In process (c), the reaction may conveniently be carried out in an organic solvent such as tetrahydrofuran, at a temperature, for example, in the range from 0 to 60° C.

Compounds of formula (II) in which A represents carbonyl may be prepared by reacting a compound of formula (X)

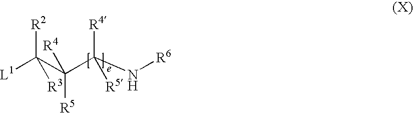

wherein L¹, e, R², R³, R⁴, R⁵, R^{4′}, R^{5′} and R⁶ are as defined in formula (II), with a compound of formula (XI)

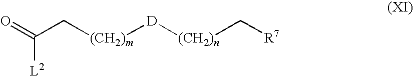

wherein L² represents a leaving group (such as hydroxyl or halogen, e.g. chlorine) and m, n, D and R⁷ are as defined in formula (II).

When L² represents hydroxyl, the reaction is conveniently carried out in the presence of an activating reagent, for example, carbonyldiimidazole or O-(7-azabenzotriazol-1-yl)-N,N,N′,N′-tetramethyluroniumhexafluorophosphate (HATU), in an organic solvent, for example, N,N-dimethylformamide or dichloromethane, at a temperature, for example in the range from 0 to 60° C.

When L² represents chlorine, the reaction is conveniently carried out in the presence of a base, for example, triethylamine or diisopropylethylamine in an organic solvent, for example, dichloromethane or tetrahydrofuran at a temperature, for example, in the range from 0 to 25° C.

Compounds of formula (II) in which A represents methylene may be prepared by contacting a corresponding compound of formula (II) in which A represents carbonyl with a reducing agent, for example, lithium aluminium hydride or borane tetrahydrofuran complex in an organic solvent, for example, tetrahydrofuran at a temperature, for example in the range from 0 to 60° C.

Compounds of formula (II) in which A represents sulphonyl may be prepared by reacting a compound of formula (X) as defined above with a compound of formula (XII)

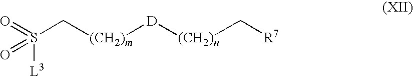

wherein L³ represents a leaving group (e.g. halogen) and m, n, D and R⁷ are as defined in formula (II). The reaction may be carried out in the presence of a base, for example, triethylamine or diisopropylethylamine in an organic solvent, for example, dichloromethane or tetrahydrofuran at a temperature, for example, in the range from 0 to 25° C.

Compounds of formula (III) may be prepared by reducing a compound of formula (XIII)

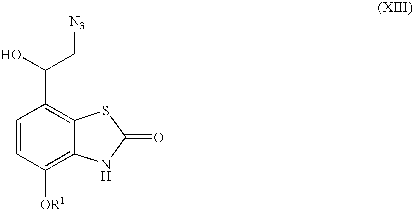

in which R¹ is as defined in formula (III), with a suitable reducing agent, for example, hydrogen in the presence of a suitable catalyst, for example, 5-10% w palladium on carbon or platininum oxide at a pressure of 1-5 atmospheres. The reaction is conveniently carried out in an organic solvent such as ethanol, methanol, ethyl acetate or tetrahydrofuran.

Compounds of formula (XIII) may be prepared by reacting a compound of formula (XIV)

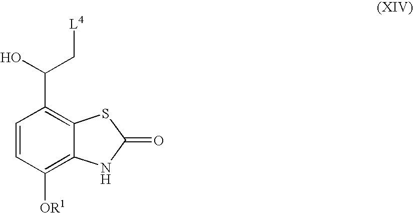

wherein L⁴ represents a leaving group (e.g. bromine, iodine, methanesulfonate or para-toluenesulfonate) and R¹ is as defined in formula (XIII), with sodium azide in the presence of, for example, sodium iodide, lithium iodide or tetrabutyl ammonium iodide. The reaction is conveniently carried out in an organic solvent, for example dimethyl sulphoxide or N,N-dimethylformamide, at a temperature, for example, in the range from 10 to 80° C., specifically from 50 to 70° C.

Compounds of formula (III) in which R¹ is hydrogen may be prepared by reacting a corresponding compound of formula (III) in which R¹ is benzyl with a suitable reducing agent, for example, hydrogen in the presence of a suitable catalyst, for example, 5-10% w palladium on carbon at a pressure of 1-5 atmospheres. The reaction is conveniently carried out in an organic solvent such as ethanol or methanol containing 5-10% w concentrated hydrochloric acid.

Compounds of formula (IV) may be prepared by treating a compound of formula (XV)

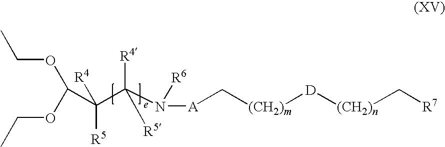

in which e, R⁴, R⁵, R^{4′}, R^{5′}, R⁶, R⁷, A, D, m and n are as defined in formula (IV), with a strong acid such as concentrated hydrochloric acid in an organic solvent such as 1,4-dioxane at a temperature, for example, of 25° C.

Compounds of formula (IV) may alternatively be prepared by oxidising a compound of formula (XVI)

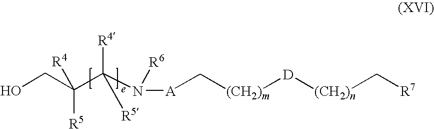

wherein e, R⁴, R⁵, R^{4′}, R^{5′}, R⁶, R⁷, A, D, m and n are as defined in formula (IV), with an oxidising agent, for example pyridinium chlorochromate or Dess-Martin periodinane in an organic solvent, for example, dichloromethane at a temperature, for example, of 25° C. Other oxidative procedures may also be employed as known to persons skilled in the art, for example, the Swern oxidation which is outlined in Synthesis, 1981, 3, 165.

Compounds of formula (V) may be prepared by reacting a compound of formula (XVII)

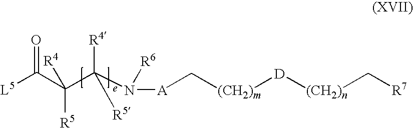

wherein L⁵ represents a leaving group (e.g. chlorine or hydroxyl) and e, R⁴, R⁵, R^{4′}, R^{5′}, R⁶, R⁷, A, D, m and n are as defined in formula (V), with a compound of formula (III) or a suitable salt thereof as defined above.

When L⁵ represents chlorine, the reaction is conveniently carried out in the presence of a base, for example, triethylamine or diisopropylethylamine in an organic solvent, for example, dichloromethane at a temperature, for example, in the range from 0 to 25° C.

When L⁵ represents hydroxyl, the reaction is conveniently carried out in the presence of an activating reagent, for example, carbonyldiimidazole or O-(7-azabenzotriazol-1-yl)-N,N,N′,N′-tetramethyluroniumhexafluorophosphate (HATU), in an organic solvent, for example, N,N-dimethylformamide or dichloromethane, at a temperature, for example in the range from 0 to 60° C.

Compounds of formula (XV) in which A represents carbonyl may be prepared by reacting a compound of formula (XVIII)

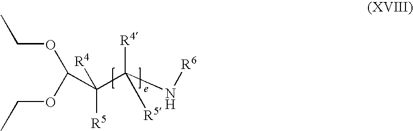

wherein e, R⁴, R⁵, R^{4′}, R^{5′} and R⁶ are as defined in formula (XV), with a compound of formula (XI) as defined above.

Compounds of formula (XV) in which A represents sulphonyl may be prepared by reacting a compound of formula (XVIII) as defined above with a compound of formula (XII) as defined above, e.g. in the presence of a base such as triethylamine or diisopropylethylamine in an organic solvent such as dichloromethane or tetrahydrofuran at a temperature, for example, in the range from 0 to 25° C.

Compounds of formula (XV) in which A represents methylene may be prepared by reacting a compound of formula (XVIII) as defined above with a compound of formula (XIX)

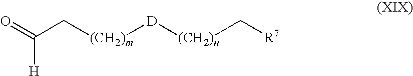

wherein m, n, D and R⁷ are as defined in formula (XV), in the presence of a reducing agent, for example, sodium cyanoborohydride or sodium triacetoxyborohydride in an organic solvent, for example, methanol, ethanol, dichloromethane, or N,N-dimethylformamide containing, for example, 0-10% w water. The reaction could also be performed in an organic solvent, for example, ethanol, acetic acid or methanol (or a combination of either) under an atmosphere of hydrogen gas with a suitable catalyst, for example, 5-10% w palladium on carbon or platinum oxide.

Compounds of formulae (XVI) and (XVII) may be prepared by processes similar to those described for the preparation of compounds of formula (XV).

Compounds of formula (XVIII) may be prepared by reacting a compound of formula (XX)

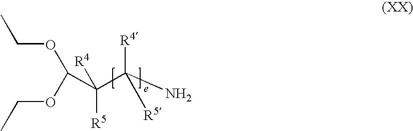

wherein e, R⁴, R⁵, R^{4′} and R^{5′} are as defined in formula (XVIII), with a compound of formula (XXI), R⁶—CHO, wherein R⁶ is as defined in formula (XVIII), in the presence of a reducing agent, for example, sodium cyanoborohydride or sodium triacetoxyborohydride in an organic solvent, for example, methanol, ethanol, dichloromethane, or N,N-dimethylformamide containing, for example, 0-10% w water. The reaction could also be performed in an organic solvent, for example, ethanol, acetic acid or methanol (or a combination of either) under an atmosphere of hydrogen gas with a suitable catalyst, for example, 5-10% w palladium on carbon or platinum oxide.

Compounds of formulae (X), (XI), (XII), (XIV), (XIX), (XX) and (XXI) are either commercially available, are known in the literature or may be prepared using known techniques.

Some of the intermediates described above are novel compounds and thus the present invention further relates to intermediate compounds of formula (III) and the intermediate compound of formula (XIII) in which R¹ represents benzyl.

Compounds of formula (I) can be converted into further compounds of formula (I) using standard procedures.

For example, compounds of formula (I) in which R¹⁰ represents a 3- to 10-membered ring system (e.g. piperidinyl) substituted by a C₁-C₆ alkoxycarbonyl substituent group may be converted to the corresponding compounds in which the ring system is unsubstituted by treating the former with, for example, trifluoroacetic acid or anhydrous hydrogen chloride, in an organic solvent such as dichloromethane or 1,4-dioxane at a temperature, for example, in the range from 15 to 30° C.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

In a further embodiment the present invention provides a compound of formula (I) having (R) absolute configuration at the asterisked (*) carbon below.

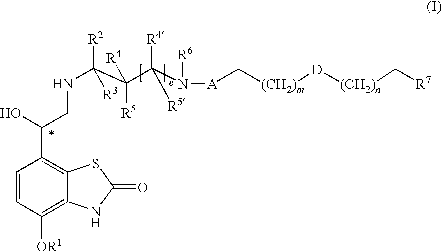

A compound of formula (I), or a pharmaceutically acceptable salt thereof, can exist a a solvate (such as a hydrate). The present invention covers such solvates.

The compounds of formula (I) and their pharmaceutically acceptable salts can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In another aspect the invention provides the use of a compound of formula (I) or a pharmaceutically acceptable salt thereof as hereinbefore defined in the manufacture of a medicament for the treatment of human diseases or conditions in which modulation of β2 adrenoreceptor activity is beneficial.

In a still further aspect the present invention provides a method of treating, or reducing the risk of, a disease or condition in which modulation of β2 adrenoreceptor activity is beneficial which comprises administering to a patient in need thereof a therapeutically effective amount of a compound of formula (I) or a pharmaceutically acceptable salt thereof as hereinbefore defined.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention still further provides a method of treating, or reducing the risk of, an inflammatory disease or condition (including a reversible obstructive airways disease or condition) which comprises administering to a patient in need thereof a therapeutically effective amount of a compound of formula (I) or a pharmaceutically acceptable salt thereof as hereinbefore defined.

In particular, the compounds of this invention may be used in the treatment of adult respiratory distress syndrome (ARDS), pulmonary emphysema, bronchitis, bronchiectasis, chronic obstructive pulmonary disease (COPD), asthma or rhinitis.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof. A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a non-steroidal glucocorticoid receptor agonist.

Where such a combination is to be administered by inhalation, then the one or more agents in addition to a compound of formula (I) can be selected from the list comprising:
a PDE4 inhibitor including an inhibitor of the isoform PDE4D;
a glucocorticoid receptor agonist, {for example a non-steroidal glucocorticoid receptor agonist, or steroidal glucocorticoid receptor agonist (such as budesonide)};
a muscarinic receptor antagonist (for example a M1, M2 or M3 antagonist, such as a selective M3 antagonist) such as ipratropium bromide, tiotropium bromide, oxitropium bromide, pirenzepine, telenzepine or a glycopyrromium bromide (such as R,R-glycopyrronium bromide or a mixture of R,S- and S,R-glycopyrronium bromide);
a modulator of chemokine receptor function (such as a CCR1 receptor antagonist); or,
an inhibitor of p38 kinase function.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples.

### General Methods

¹H NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B 1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:

HPLC: High performance liquid chromatography

### EXAMPLE 1

### N-[2-[2-(4-Benzyloxy-2-oxo-3H-benzothiazol-7-yl)-(2R)-2-hydroxy-ethyl]aminoethyl]-N-butyl-3-phenethyloxy-propanamide

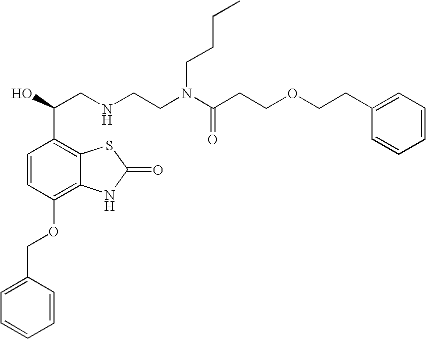

### a) N-Butyl-N-(2-hydroxy-ethyl)-3-phenethyloxy-propionamide

To a stirred solution of tert-butyl-3-phenethyloxypropionate (2.5 g) in dichloromethane (10 mL) was added trifluoroacetic acid (5 mL) and the mixture stirred for 4 hours and was then concentrated. The residue was dissolved in dichloromethane (10 mL) and oxalyl chloride (3 mL) added. The reaction was stirred for 2 hours and concentrated. The residue was dissolved in dichloromethane (20 mL) and added to a solution of N,N-diisopropylethylamine (3.23 g) and 2-butylaminoethanol (1.41 g) in dichloromethane (20 mL) at 5° C. The reaction was stirred for 1 hour and poured into 2N hydrochloric acid (30 mL) and extracted into ethyl acetate (2×30 mL). The organic phase was dried with anhydrous magnesium sulphate, filtered and concentrated to give the sub-titled compound (1.95 g) as an oil.

¹H NMR (400 MHz, CDCl₃) δ 7.30-7.18 (m, 5H), 3.83-3.74 (m, 3H), 3.68 (t, 3H), 3.53-3.45 (m, 3H), 3.35-3.25 (m, 2H), 2.89-2.85 (m, 2H), 2.67-2.60 (m, 2H), 1.58-1.47 (m, 2H), 1.36-1.27 (m, 2H), 0.96-0.91 (m, 3H).

### b) N-Butyl-N-(2-oxo-ethyl)-3-phenethyloxy-propionamide

To a stirred solution of N-butyl-N-(2-hydroxy-ethyl)-3-phenethyloxy-propionamide (Example 1a), 0.45 g) in dichloromethane (30 mL) was added Dess-Martin Periodinane (0.65 g). After 3 hours at room temperature a white solid was filtered off and the organics were washed with saturated sodium bicarbonate solution (2×10 mL) and then brine (20 mL), dried with anhydrous magnesium sulphate, filtered and concentrated. Purification by flash column chromatography (eluting with 20% ethyl acetate/hexane to 100% ethyl acetate) gave the sub-titled compound (0.29 g).

¹H NMR (300 MHz, CDCl₃) δ 9.47 (s, 1H), 7.31-7.18 (m, 5H), 4.01 (s, 2H), 3.77 (t, 2H), 3.69 (t, 2H), 3.32 (t, 2H), 2.88 (t, 2H), 2.68 (t, 2H), 1.57-1.47 (m, 2H), 1.33-1.26 (m, 2H), 0.94 (t, 3H).

### c) 7-((1R)-2-Azido-1-hydroxy-ethyl)-4-benzyloxy-3H-benzothiazol-2-one

To a solution of 4-(benzyloxy)-7-[(1R)-2-bromo-1-hydroxyethyl]-1,3-benzothiazol-2(3H)-one (prepared as described in WO 2004/016578, 340 mg) in dimethyl sulfoxide (8 mL) was added sodium azide (231 mg) and sodium iodide (147 mg). The reaction mixture was heated at 65° C. for 5 hours. At the end of this time the mixture was cooled and then partitioned between ethyl acetate and water, the organic phase was washed with water, dried with anhydrous magnesium sulphate, filtered and concentrated under reduced pressure. The residue was purified by flash chromatography on silica gel, eluting with 20% ethyl acetate in toluene to yield the sub-titled compound (195 mg).

¹H NMR (400 MHz, D₆-DMSO) δ 11.89 (s, 1H), 7.54 (d, 2H), 7.38 (t, 2H), 7.33-7.29 (m, 1H), 7.02 (s, 2H), 6.13 (d, 1H), 5.25 (s, 2H), 4.81-4.77 (m, 1H), 3.40-3.27 (m, 2H).

### d) 7-((1R)-2-Amino-1-hydroxy-ethyl)-4-benzyloxy-3H-benzothiazol-2-one

A solution of 7-((1R)-2-Azido-1-hydroxy-ethyl)-4-benzyloxy-3H-benzothiazol-2-one (Example 1c), 195 mg) in a mixture of ethanol (8 mL) and tetrahydrofuran (4 mL) was treated with 10% palladium on carbon (20 mg) and the resulting mixture stirred vigorously under 3 atmospheres pressure of hydrogen gas for 20 hours. The catalyst was filtered off and the solvent removed under reduced pressure. The residue was purified by flash chromatography on silica gel eluting with 1% concentrated aqueous ammonia and 12% methanol in dichloromethane. The resultant product was dissolved in 1,4-dioxane and treated drop-wise with a 4 molar solution of hydrogen chloride in 1,4 dioxane (0.5 mL), evaporation of the solvent under reduced pressure yielded the sub-titled compound (160 mg).

m/e 315 (M−H)⁺

¹H NMR (400 MHz, D₆-DMSO) δ 8.01 (s, 2H), 7.55 (d, 2H), 7.39 (t, 2H), 7.31 (t, 1H), 7.04 (q, 2H), 6.39 (d, 1H), 5.26 (s, 2H), 4.83 (dt, 1H), 2.97-2.83 (m, 2H).

### e) N-[2-[2-(4-Benzyloxy-2-oxo-3H-benzothiazol-7-yl)-2-hydroxy-ethyl]aminoethyl]-N-butyl-3-phenethyloxy-propanamide

A solution of 7-((1R)-2-amino-1-hydroxy-ethyl)-4-benzyloxy-3H-benzothiazol-2-one hydrochloride (Example 1d), 0.20 g) in methanol (10 mL) was treated with N-butyl-N-(2-oxo-ethyl)-3-phenethyloxy-propionamide (Example 1b), 0.165 g) in methanol/dichloromethane (1 mL/0.1 mL). Acetic acid (5 drops) was added and the resulting mixture stirred at room temperature for 10 minutes and was then treated with sodium cyanoborohydride (0.021 g). The reaction mixture was stirred at room temperature for 20 hours, the solvent was removed under reduced pressure and the residue partitioned between dilute aqueous ammonia and ethyl acetate. The organic phase was washed with water, dried with anhydrous magnesium sulphate, filtered and concentrated under reduced pressure. The residue was purified by flash chromatography on silica gel, eluting with 1% concentrated aqueous ammonia and 5% methanol in dichloromethane to yield the titled compound as a clear oil (0.11 g).

m/e 593 (M+H)⁺

¹H NMR (400 MHz, D₆-DMSO, 90° C.) δ 8.63 (s, 1H), 7.52 (d, 2H), 7.37 (t, 3H), 7.31 (d, 2H), 7.29-7.14 (m, 5H), 7.03 (dd, 2H), 6.28 (s, 1H), 5.24 (s, 2H), 4.94 (d, 1H), 3.65 (t, 4H), 3.60 (t, 5H), 3.25 (t, 2H), 2.78 (t, 2H), 2.54 (t, 2H), 2.49-2.48 (m, 4H), 1.32-1.22 (m, 2H), 0.90 (t, 3H).

### EXAMPLE 2

### N-Butyl-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide Hydrochloride

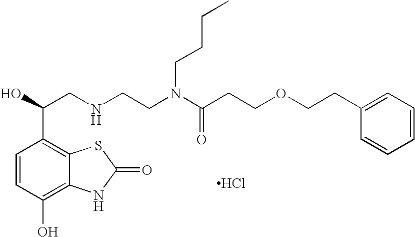

A solution of N-[2-[2-(4-benzyloxy-2-oxo-3H-benzothiazol-7-yl)-2-hydroxy-ethyl]aminoethyl]-N-butyl-3-phenethyloxy-propanamide (Example 1, 0.11 g) in 98% formic acid (3 mL) was treated with palladium black (10 mg) and the mixture stirred vigorously at room temperature under nitrogen. Further 10 mg aliquots of palladium black were added at 30 minute intervals over a 5 hour period. At the end of this time the mixture was filtered and the resultant solution was evaporated under reduced pressure. The residue was purified by reverse phase HPLC using a gradient elution of 5% to 75% acetonitrile in 0.2% aqueous ammonium acetate. The isolated product was dissolved in 4 N hydrogen chloride in 1,4-dioxane and concentrated. The oily residue was triturated with ether, decanting the ether and concentrating to afford the titled compound as a white solid (0.027 g).

m/e 502 (M+H⁺)

¹H NMR (400 MHz, CD₃OD) δ 7.25-7.17 (m, 5H), 7.00 (d, 1H), 6.76 (d, 1H), 4.98-4.95 (m, 1H), 3.75 (t, 2H), 3.68-3.63 (m, 4H), 3.35-3.30 (m, 2H), 3.18-3.11 (m, 4H), 2.83 (t, 2H), 2.62 (t, 2H), 1.59-1.53 (m, 2H), 1.39-1.28 (m, 2H), 0.97 (t, 3H).

### EXAMPLE 3

### N-[2-({(2R)-2-[4-(Benzyloxy)-2-oxo-2,3-dihydro-1,3-benzothiazol-7-yl]-2-hydroxyethyl}amino)ethyl]-3-(2-phenylethoxy)-N-(2-phenylethyl)propanamide

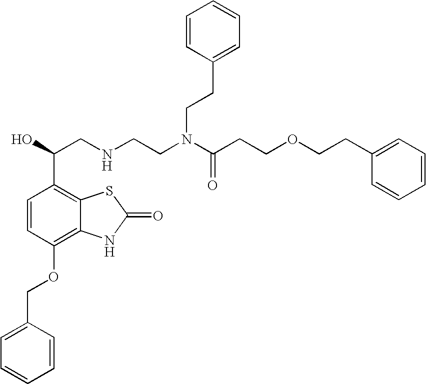

### a) N-(2,2-Diethyloxyethyl)-N-phenethyl-3-phenethyloxy-propanamide

3-Phenethyloxypropanoic acid (0.34 g) was dissolved in dichloromethane (20 mL) and treated with oxalyl chloride (0.32 g) and a drop of dimethylformamide at ambient temperature. The resultant solution was stirred at ambient temperature for 2 hours. The solution was then concentrated and azeotroped with dichloromethane (2×20 mL). The collected residue was dissolved in dichloromethane and added portion-wise to a stirred solution of 2,2-diethoxy-N-phenethyl-ethanamine (0.41 g) and triethylamine (0.6 mL), dissolved in dichloromethane (20 mL). The resulting solution was stirred at room temperature for 2 hours. The mixture was then concentrated, taken up in ethyl acetate (50 mL), washed with water (2×25 mL), brine (50 mL), dried over anhydrous magnesium sulphate, filtered and concentrated to give the sub-titled compound (0.53 g) as a viscous oil.

m/e 414.0 [M+H]⁺

¹H NMR (400 MHz, CDCl₃) δ 7.29-7.13 (m, 10H), 4.66 & 4.48 (t, 1H), 3.79-3.39 (m, 12H), 2.94-2.81 (m, 4H), 2.67 & 2.45 (t, 2H), 1.22-1.16 (m, 6H).

### b) N-(2-Oxoethyl)-N-phenethyl-3-phenethyloxy-propanamide

N-(2,2-Diethyloxyethyl)-N-phenethyl-3-phenethyloxy-propanamide (Example 3a), 0.23 g) was dissolved in dioxane (5 mL) and treated with concentrated hydrochloric acid (1.5 mL) and stirred at ambient temperature for 2.5 hours. The reaction mixture was then poured into dichloromethane (20 mL), washed with water (2×20 mL) and brine (20 mL). The organic layer was isolated, dried over anhydrous magnesium sulphate, filtered and concentrated to give the sub-titled compound (0.13 g) as a viscous oil.

m/e 340.0 [M+H]⁺

¹H NMR (400 MHz, CDCl₃) δ 9.42 (s, 1H), 7.33-7.14 (m, 110H), 3.92 (s, 2H), 3.78-3.57 (m, 6H), 2.90-2.81 (m, 4H), 2.53 & 2.40 (t, 2H).

### c) N-[2-({(2R)-2-[4-(Benzyloxy)-2-oxo-2,3-dihydro-1,3-benzothiazol-7-yl]-2-hydroxyethyl}amino)ethyl]-3-(2-phenylethoxy)-N-(2-phenylethyl)propanamide

A solution of 7-((1R)-2-amino-1-hydroxy-ethyl)-4-benzyloxy-3H-benzothiazol-2-one hydrochloride (Example 1d), 0.20 g) in methanol (5 mL) was treated with N-(2-oxoethyl)-N-phenethyl-3-phenethyloxy-propanamide (Example 3b), 0.26 g) in methanol (2 mL). Acetic acid (5 drops) was added and the resulting mixture was stirred at room temperature for 10 minutes and then treated with sodium cyanoborohydride (0.021 g). The reaction mixture was stirred at room temperature for 20 hours and at the end of this time the solvent was removed under reduced pressure and the residue was partitioned between dilute aqueous ammonia and ethyl acetate. The organic phase was washed with water, dried with anhydrous magnesium sulphate, filtered and concentrated under reduced pressure to give a crude product (0.34 g). The residue was purified by flash chromatography on silica gel, eluting with 1% concentrated aqueous ammonia and 5% methanol in dichloromethane to yield the titled compound (0.13 g).

m/e 640 (M+H⁺, 100%)

¹H NMR (400 MHz, CDCl₃) δ 7.41-7.38 (m, 4H), 7.31-7.14 (m, 11H), 7.00-6.98 (m, 1H), 6.83-6.80 (m, 1H), 5.12 (s, 2H), 4.70-4.61 (m, 1H), 3.81 (t, 1/2H), 3.73-3.62 (m, 3H), 3.53-3.49 (m, 2H), 3.44-3.42 (m, 1H), 3.30-3.21 (m, 1/2H), 2.88-2.62 (m, 1H), 2.46 (t, 1H), 1.26 (s, 2H).

### EXAMPLE 4

### N-(2-{[(2R)-2-Hydroxy-2-(4-hydroxy-2-oxo-2,3-dihydro-1,3-benzothiazol-7-yl)ethyl]amino}ethyl)-3-(2-phenylethoxy)-N-(2-phenylethyl)propanamide

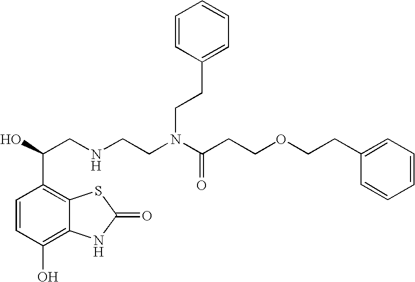

A solution of N-[2-({(2R)-2-[4-(benzyloxy)-2-oxo-2,3-dihydro-1,3-benzothiazol-7-yl]-2-hydroxyethyl}amino)ethyl]-3-(2-phenylethoxy)-N-(2-phenylethyl)propanamide (Example 3, 0.12 g) in 98% formic acid (5 mL) was treated with palladium black (10 mg) and the mixture stirred vigorously at room temperature under nitrogen. Further 10 mg aliquots of palladium black were added at 30 minute intervals over a 5 hour period. At the end of this time the mixture was filtered and the resulting solution evaporated under reduced pressure. The residue was purified by flash chromatography on silica gel, eluting with 1% concentrated aqueous ammonia and 10% methanol in dichloromethane to yield the titled compound (0.023 g).

m/e 550 (M+H⁺, 100%)

¹H NMR (400 MHz, CD₃OD) δ 7.28-7.16 (m, 10H), 6.92 (dd, 1H), 6.71 (d, 1H), 4.79-4.70 (m, 1H), 3.72-3.49 (m, 8H), 3.43 (t, 1H), 2.85-2.68 (m, 8H), 2.58 (t, 1H), 2.36 (t, 1H).

### EXAMPLE 5

### N-(2-{[(2R)-2-Hydroxy-2-(4-hydroxy-2-oxo-2,3-dihydro-1,3-benzothiazol-7-yl)ethyl]amino}ethyl)-N-methyl-3-(2-phenylethoxy)propanamide Trifluoroacetate

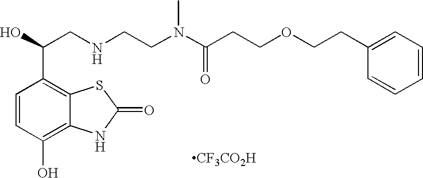

### a) N-(2,2-Dimethoxy-ethyl)-N-methyl-3-phenethyloxy-propionamide

A solution of 3-(2-phenylethoxy)-propanoic acid (1.8 g) in dichloromethane (30 mL) was treated with oxalyl chloride (2 mL) followed by N,N-dimethylformamide (1 drop) and the resulting mixture stirred for 4 hours at room temperature. The solvent was then removed under reduced pressure and the residue azeotroped three times with dichloromethane. The residue was then added, drop-wise to a solution of methylaminoacetaldehyde dimethyl acetal (1.13 g) and triethylamine (3.08 mL) in dichloromethane (50 mL). This mixture was stirred for 1 hour at room temperature. At the end of this time the reaction mixture was washed with water and the organic phase was dried over magnesium sulphate, filtered and the solvent was removed under reduced pressure to yield the sub-titled compound (2.6 g).

m/e 296 (M+H)⁺

### b) N-Methyl-N-(2-oxo-ethyl)-3-phenethyloxy-propionamide

To a solution of N-(2,2-dimethoxy-ethyl)-N-methyl-3-phenethyloxy-propionamide (Example 5a, 0.9 g) in 1,4-dioxane (5 mL) was added concentrated aqueous hydrochloric acid (5 mL) and the solution was allowed to stand at room temperature for 15 minutes. At the end of this time the reaction mixture was partitioned between water (100 mL) and dichloromethane (100 mL). The aqueous layer was re-extracted with dichloromethane (2×25 mL) and the combined organic phases were then washed with water, dried over anhydrous magnesium sulphate, filtered and evaporated under reduced pressure. The crude product was purified by flash chromatography on a silica column, eluting with 10% iso-hexane in ethyl acetate to yield the sub-titled compound (0.43 g).

m/e 248 (M−H⁺, 100%)

### c) N-[2-({(2R)-2-[4-(Benzyloxy)-2-oxo-2,3-dihydro-1,3-benzothiazol-7-yl]-2-hydroxyethyl}amino)ethyl]-N-methyl-3-(2-phenylethoxy)propanamide

The sub-titled compound was prepared from 7-((1R)-2-amino-1-hydroxy-ethyl)-4-benzyloxy-3H-benzothiazol-2-one hydrochloride (Example 1d), 250 mg) and N-methyl-N-(2-oxo-ethyl)-3-phenethyloxy-propionamide (Example 5b), 77 mg) using the method of Example 1e). The crude product was purified by flash chromatography on a silica column, eluting with 1% concentrated aqueous ammonia and 8% methanol in dichloromethane to yield the sub-titled compound (140 mg).

m/e 550 (M+H⁺, 100%)

### d) N-(2-{[(2R)-2-Hydroxy-2-(4-hydroxy-2-oxo-2,3-dihydro-1,3-benzothiazol-7-yl)ethyl]amino}ethyl)-N-methyl-3-(2-phenylethoxy)propanamide Trifluoroacetate

The titled compound was prepared from N-[2-({(2R)-2-[4-(benzyloxy)-2-oxo-2,3-dihydro-1,3-benzothiazol-7-yl]-2-hydroxyethyl}amino)ethyl]-N-methyl-3-(2-phenylethoxy)propanamide (Example 5c), 130 mg) using the method of Example 2. The crude product was purified by reverse phase HPLC using a gradient elution of 5% to 75% acetonitrile in 0.2% aqueous trifluoroacetic acid to yield the titled compound (32 mg).

m/e 460 (M+H)⁺

¹H NMR (400 MHz, D₆-DMSO) δ 11.66 (d, 1H), 10.22 (d, 1H), 8.71-8.57 (m, 2H), 7.29-7.16 (m, 5H), 6.91 (d, 1H), 6.79-6.76 (m, 1H), 6.49-6.43 (m, 1H), 4.87-4.84 (m, 1H), 3.64-3.52 (m, 6H), 3.08 (s, 4H), 2.95 (s, 2H), 2.80-2.77 (m, 3H), 2.59-2.55 (m, 2H).

### EXAMPLE 6

### N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-[2-(1-naphthyl)ethoxy]propanamide

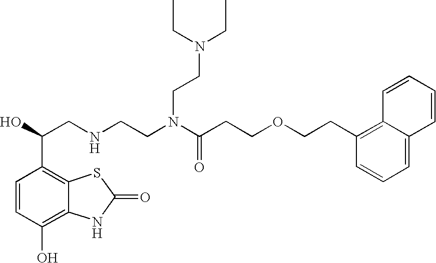

### a) tert-Butyl 3-[2-(1-naphthyl)ethoxy]propanoate

1-Naphthalene ethanol (10 g) was treated with benzyltrimethylammonium hydroxide (Triton B^{(R)}) (0.9 mL of a 40% solution in methanol) and the resulting mixture was stirred in vacuo for 30 minutes. The mixture was then cooled to 0° C. and treated with tert-butyl acrylate (8.19 g). The resulting mixture was slowly warmed to room temperature and stirred overnight. The crude mixture was subsequently absorbed onto aluminium oxide (30 g) and eluted with diethyl ether (200 mL). The organics were concentrated to give a crude material (16.6 g) which was purified by flash silica chromatography eluting with 1:8, diethylether:hexane to give the sub-titled compound (12.83 g).

¹H NMR (300 MHz, CDCl₃) δ 8.05 (dd, 1H), 7.84 (dd, 1H), 7.72 (dd, 1H), 7.54-7.34 (m, 4H), 3.81-3.69 (m, 4H), 3.35 (t, 2H), 2.52-2.47 (m, 2H), 1.45 (s, 9H).

### b) 3-[2-(1-Naphthyl)ethoxy]propanoic Acid

tert-Butyl 3-[2-(1-naphthyl)ethoxy]propanoate (Example 6a), 6.19 g) was taken up in dichloromethane (30 mL) and treated with trifluoroacetic acid (5 mL). The resulting solution was stirred at room temperature for 2 hours, an additional 1 mL of trifluoroacetic acid was added and the solution was stirred overnight. The mixture was concentrated, taken up in 2M sodium hydroxide solution (30 mL) and washed with ether (2×20 mL). The aqueous layer was subsequently acidified (using 1M hydrochloric acid) and extracted with ether (2×30 mL). The combined organics were washed with brine (20 mL), dried with anhydrous magnesium sulphate, filtered and concentrated in vacuo to give the sub-titled compound (5.66 g) as a clear oil.

¹H NMR (300 MHz, CDCl₃) δ 8.05 (bs, 1H), 7.85 (bs, 1H), 7.74 (bs, 1H), 7.50-7.38 (m, 4H), 3.84-3.75 (bm, 4H), 3.39 (bs, 2H), 2.65 (bs, 2H).

### c) N-(2-Diethylaminoethyl)-N-(2-hydroxyethyl)-3-[2-(1-naphthyl)ethoxy]propanamide

Oxalyl chloride (0.33 g) was added dropwise to a solution of 3-[2-(1-naphthyl)ethoxy]propanoic acid (Example 6b), 0.53 g) in dichloromethane (10 mL), N,N-dimethylformamide

(1 drop) was added and stirring was continued at room temperature for 1 hour. The mixture was subsequently concentrated, dissolved in dichloromethane (10 mL) and added dropwise to a solution of 2-(2-diethylaminoethylamino)ethanol (0.35 g) and diisopropylethylamine (0.56 g) in dichloromethane (10 mL). The resulting mixture was stirred at room temperature for 1 hour, diluted (dichloromethane, 50 mL), washed with water (2×20 mL) and then brine (20 mL), dried over magnesium sulfate and concentrated. The residue was purified by flash column chromatography (eluting with 5-7% methanol in dichloromethane) to give 0.63 g of the sub-titled compound.

¹H NMR (400 MHz, CDCl₃) δ 8.05 (d, 1H), 7.85 (d, 1H), 7.73 (d, 1H), 7.52-7.47 (m, 2H), 7.42-7.35 (m, 2H), 3.84-3.78 (m, 6H), 3.72-3.70 (m, 1/2H), 3.45-3.35 (m, 6H), 2.79-2.77 (m, 1+1/2H), 2.62-2.58 (m, 2H), 2.54-2.49 (m, 4H), 1.04-1.01 (m, 6H).

### d) 7-[(1R)-2-Amino-1-hydroxyethyl]-4-hydroxy-1,3-benzothiazol-2(3H)-one Hydrochloride

A solution of 7-((1R)-2-amino-1-hydroxy-ethyl)-4-benzyloxy-3H-benzothiazol-2-one hydrochloride (Example 1d), 1.8 g) in methanol (60 mL) and concentrated hydrochloric acid (4 mL) was stirred vigorously in the presence of 110% w palladium on carbon catalyst (0.36 g) and under 4 atmospheres pressure of hydrogen gas for 2 hours. Further 10% w palladium on carbon catalyst (0.24 g) was added and stirring continued under hydrogen for 1 hour. The catalyst was filtered off and the solvent evaporated under reduced pressure to yield the sub-titled compound (1.3 g).

m/e 227 (M+H)⁺

¹H NMR (400 MHz, D₆-DMSO) δ 11.70 (s, 1H), 10.21 (s, 1H), 8.04 (s, 3H), 6.92 (d, 1H), 6.79 (d, 1H), 6.32 (d, 1H), 4.81-4.79 (m, 1H), 2.90-2.81 (m, 2H).

### e) N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-[2-(1-naphthyl)ethoxy]propanamide

A solution of dimethyl sulfoxide (0.097 g) in dichloromethane (1 mL) was added to oxalyl chloride (0.079 g) in dichloromethane (10 mL) at −78° C. The reaction was stirred for 15 minutes, treated with N-(2-diethylaminoethyl)-N-(2-hydroxyethyl)-3-[2-(1-naphthyl)ethoxy]propanamide (Example 6c), 0.22 g) in dichloromethane (1 mL+1 mL wash), and stirred for a further 15 minutes. Triethylamine (0.29 g) was added and the reaction was allowed to warm to room temperature over 1 hour. The mixture was subsequently diluted (dichloromethane 30 mL), the organics washed with saturated sodium bicarbonate solution (20 mL) and brine (20 mL), dried with anhydrous magnesium sulphate, filtered and concentrated in vacuo. The crude product was dissolved in methanol (15 mL) and 7-[(1R)-2-amino-1-hydroxyethyl]-4-hydroxy-1,3-benzothiazol-2(3H)-one hydrochloride (Example 6d), 0.108 g) was added along with acetic acid (0.1 mL) and water (0.1 mL). After stirring at room temperature for 20 minutes, sodium cyanoborohydride (0.020 g) was added and the reaction mixture was stirred for 4 hours. Ammonia (7N in methanol, 1 mL) was added and the mixture was concentrated. The crude residue was purified by flash column chromatography eluting with 1% ammonia; 5%-7% methanol in dichloromethane to afford the titled compound as a white solid (0.029 g).

m/e 595 (M+H)⁺

¹H NMR (300 MHz, CD₃OD) δ 8.08 (d, 1H), 7.83 (d, 1H), 7.71 (bs, 1H), 7.51-7.38 (bm, 4H), 6.96-6.92 (m, 1H), 6.74 (d, 1H), 4.79-4.74 (m, 1H), 3.84-3.78 (m, 4H), 3.45-3.27 (m, 6H), 2.83-2.49 (m, 12H), 1.10-0.99 (m, 6H)

### EXAMPLE 7

### N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide

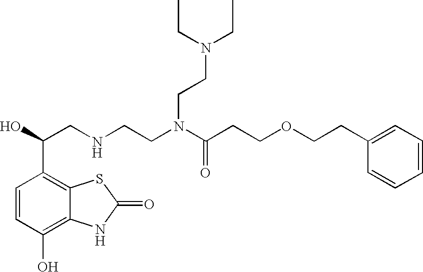

### a) N-(2-Diethylaminoethyl)-N-(2-hydroxyethyl)-3-phenethyloxy-propanamide

Oxalyl chloride (0.23 g) was added dropwise to a solution of 3-phenethyloxypropanoic acid (0.32 g) in dichloromethane (10 mL), N,N-dimethylformamide (1 drop) was added and stirring was continued at room temperature for 1 hour. The mixture was subsequently concentrated, redissolved in dichloromethane (10 mL) and added dropwise to a solution of 2-(2-diethylaminoethylamino)ethanol (0.26 g) and diisopropylethylamine (0.42 g) in dichloromethane (10 mL). The resulting mixture was stirred at room temperature for 1 hour and then diluted (dichloromethane, 50 mL). The organics were washed with water (2×20 mL) and brine (20 mL), dried over magnesium sulfate and concentrated to give the crude sub-titled product (0.39 g).

¹H NMR (400 MHz, CDCl₃) δ 7.29-7.19 (m, 5H), 3.80-3.64 (m, 6H), 3.47-3.40 (m, 4H), 2.89-2.77 (m, 4H), 2.64-2.49 (m, 6H), 1.04 (s, 6H)

### b) N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide

A solution of dimethyl sulfoxide (0.076 g) in dichloromethane (1 mL) was added to a solution of oxalyl chloride (0.062 g) in dichloromethane (10 mL) at −78° C. The reaction was stirred for 15 minutes and then a solution of N-(2-diethylaminoethyl)-N-(2-hydroxyethyl)-3-phenethyloxy-propanamide (Example 7a), 0.15 g) in dichloromethane (1 mL+1 mL wash) was added and the reaction mixture was stirred for a further 15 minutes. Triethylamine (0.23 g) was added and the reaction mixture was allowed to warm to room temperature over 1 hour. The mixture was subsequently diluted (dichloromethane 30 mL) and the organics were washed with saturated sodium bicarbonate solution (20 mL) followed by brine (20 mL), dried with anhydrous magnesium sulphate, filtered and concentrated in vacuo. The crude product was dissolved in methanol (10 mL) and 7-[(1R)-2-amino-1-hydroxyethyl]-4-hydroxy-1,3-benzothiazol-2(3H)-one hydrochloride (Example 6d), 0.094 g) was added along with acetic acid (0.1 mL) and water (0.1 mL). After stirring at room temperature for 30 minutes, sodium cyanoborohydride (0.013 g) was added and the reaction mixture was stirred overnight. Ammonia (7N in methanol, 1 mL) was added and the mixture was concentrated. The crude residue was purified by flash column chromatography eluting with 1% ammonia; 7% methanol in dichloromethane to afford the titled compound as a white solid (0.013 g).

m/e 545 (M+H)⁺

¹H NMR (400 MHz, D₆-DMSO D₆) δ 7.27-7.17 (m, 5H), 6.85 (d, 1H), 6.69 (d, 1H), 4.58 (bs, 1H), 3.62-3.17 (m, 12H), 2.77 (t, 2H), 2.66 (t, 2H), 2.51-2.44 (m, 8H), 0.95-0.91 (m, 6H).

### EXAMPLE 8

### 4-Hydroxy-7-{(1R)-1-hydroxy-2-[(2-{[3-(2-phenylethoxy)propyl]amino}-ethyl)amino]ethyl}-1,3-benzothiazol-2(3H)-one

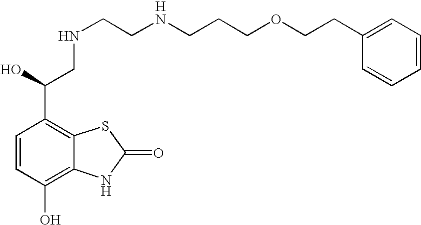

### a) N-(2-Hydroxy-ethyl)-3-phenethyloxy-propionamide

To a solution of tert-butyl-3-phenethyloxypropionate (2.5 g) in dichloromethane (10 mL) was added trifluoroacetic acid (10 mL) and the mixture was stirred for 20 hours and concentrated. The residue was dissolved in dichloromethane (10 mL) and oxalyl chloride (5 mL) was added. The mixture was stirred for 2 hours and concentrated. The residue was dissolved in dichloromethane (10 mL) and added to a solution of ethanolamine (0.915 g) and triethylamine (3.033 g) in dichloromethane (20 mL) at 0° C. The mixture was stirred for 1 hour and was poured into 2N hydrochloric acid and extracted into ethyl acetate (3×30 mL). The organic phase was dried with anhydrous magnesium sulphate, filtered and concentrated and the residue obtained was purified by chromatography on silica gel eluting with ethyl acetate to afford the sub-titled compound (2.1 g) as an oil.

¹H NMR (400 MHz, CDCl₃) δ 7.33-7.22 (m, 5H), 6.56 (s, 1H), 3.72 (t, 2H), 3.67 (t, 2H), 3.56 (t, 2H), 3.18 (q, 2H), 2.90 (t, 2H), 2.46 (t, 2H).

### b) 2-(3-Phenethyloxy-propylamino)-ethanol

To a solution of N-(2-hydroxy-ethyl)-3-phenethyloxy-propionamide (Example 8a), 2 g) in tetrahydrofuran (10 mL) was added borane-tetrahydrofuran complex (25 mL of a 1 M solution in tetrahydrofuran) and the resulting mixture was stirred at 60° C. for 3 hours. The reaction was cooled and methanol (10 mL) was cautiously added. The reaction mixture is was concentrated and the residue obtained was dissolved in methanol (10 mL) and was purified by using a solid phase extraction with a sulfonic acid sorbent to afford the sub-titled compound (0.7 g).

¹H NMR (400 MHz, CDCl₃) δ 7.31-7.26 (m, 3H), 7.23-7.21 (m, 2H), 3.64 (t, 2H), 3.59 (t, 2H), 3.51 (t, 2H), 2.88 (t, 2H), 2.73 (t, 2H), 2.68 (t, 2H), 2.01 (s, 2H), 1.75 (quintet, 2H).

### c) (2-Hydroxy-ethyl)-(3-phenethyloxy-propyl)-carbamic acid tert-butyl Ester

To a solution of 2-(3-phenethyloxy-propylamino)-ethanol (Example 8b), 0.9 g) in dichloromethane (5 mL) was added di-tert-butyl dicarbonate (0.967 g) and the mixture was stirred for one hour, concentrated and the residue obtained was purified by chromatography on silica gel eluting with ethyl acetate/iso-hexane (1/1) to afford the sub-titled compound as an oil (1.247 g).

¹H NMR (300 MHz, CDCl₃) δ 7.31-7.18 (m, 5H), 3.70-3.59 (m, 4H), 3.51-3.42 (m, 2H), 3.30 (t, 2H), 2.87 (t, 2H), 1.85-1.77 (m, 2H), 1.46 (s, 9H).

### d) 4-Hydroxy-7-{(1R)-1-hydroxy-2-[(2-{[3-(2-phenylethoxy)propyl]amino}ethyl)-amino]ethyl}-1,3-benzothiazol-2(3H)-one

To a stirred solution of dimethyl sulfoxide (0.234 g) in dichloromethane (5 mL) was added oxalyl chloride (0.253 g) at −70° C. The mixture was stirred for 15 minutes and then (2-hydroxy-ethyl)-(3-phenethyloxy-propyl)-carbamic acid tert-butyl ester (Example 8c), 0.323 g) in dichloromethane (2 mL) was added. The reaction mixture was stirred for 30 minutes and then triethylamine (0.505 g) was added. The reaction mixture was stirred at −70° C. for 15 minutes and was allowed to warm to room temperature with stirring over a one hour period. The mixture was poured into pH 7.4 phosphate buffer and was extracted into dichloromethane (2×25 mL) and the combined organics were concentrated and the residue obtained was dissolved in methanol (30 mL) and concentrated once more. The residue was obtained was again dissolved in methanol (10 mL) and 7-[(1R)-2-amino-1-hydroxyethyl]-4-hydroxy-1,3-benzothiazol-2(3H)-one hydrochloride (Example 6d), 0.1312 g) was added along with acetic acid (0.1 mL). The mixture was stirred for 30 minutes and sodium cyanoborohydride (0.031 g) was added. The reaction mixture was stirred for 4 hours, concentrated aqueous ammonia (0.5 mL) was added and the mixture was concentrated. The residue obtained was purified by chromatography on silica eluting with methanol in dichloromethane+1% ammonia on a gradient of 2-20%. The fractions containing the BOC (t-butyloxycarbonyl)-protected compound were concentrated and dissolved in dichloromethane (2 mL) and trifluoroacetic acid (2 mL) was added. The reaction was stirred for 3 hours and concentrated. The residue was purified by reverse phase HPLC on a gradient elution of 5-75% methanol in 0.2% w ammonia to afford the titled compound (0.06 g) as a white solid.

m/e 432 (M+H)⁺

¹H NMR (400 MHz, D₆-DMSO) δ 7.28-7.15 (m, 5H), 6.83 (d, 1H), 6.66 (d, 1H), 4.56 (q, 1H), 3.55 (t, 2H), 3.41 (t, 2H), 2.79 (t, 2H), 2.68-2.53 (m, 8H), 1.60 (quintet, 2H).

### EXAMPLE 9

### 3-[2-(3-Chlorophenyl)ethoxy]-N-[2-(diethylamino)ethyl]-N-(2-{[(2R)-2-hydroxy-2-(4-hydroxy-2-oxo-2,3-dihydro-1,3-benzothiazol-7-yl)ethyl]amino}ethyl)propanamide

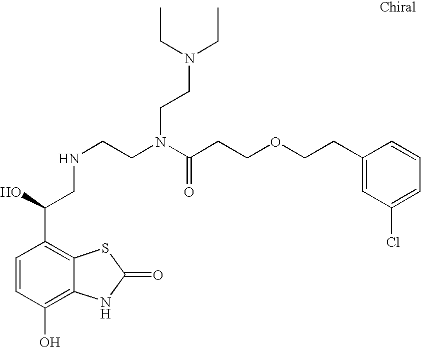

### a) tert-Butyl 3-[2-(3-chlorophenyl)ethoxy]propanoate

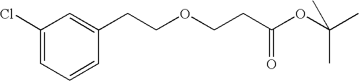

2-(3-Chlorophenyl)ethanol (3.0 g, 19.2 mmol) was treated with Triton B (290 μl) (40 wt % in methanol). The methanol was removed by evaporation and the residue azeotroped with toluene (×2). The mixture was cooled in an ice bath and tert-butyl acrylate (4.13 ml) was added slowly. The mixture was stirred for 4 days and then evaporated. The residue was purified by chromatography on silica with 5% ethyl acetate/isohexane to give the sub-title compound as a clear, colourless oil (5.25 g).

1H NMR (300 MHz, DMSO-d₆) δ 1.39 (s, 9H), 2.42 (t, 2H), 2.82 (t, 2H), 3.58 (m, 4H), 7.20-7.34 (m, 4H).

### b) 3-[2-(3-Chlorophenyl)ethoxy]propanoic Acid

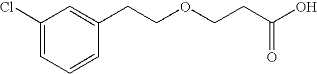

The product from part a) (5.25 g) was dissolved in DCM (100 ml), and TFA (40 ml) was added. After stirring for 2 hr the solvent was removed in vacuo to give the sub-title compound as a purple oil (4.81 g).

¹H NMR (400 MHz, DMSO-d₆) δ 2.42 (t, J=6.6 Hz, 2H), 2.79 (t, J=6.6 Hz, 2H), 3.58 (q, J=6.5 Hz, 4H), 7.19-7.31 (m, 4H).

### c) N′-(2,2-Dimethoxyethyl)-N,N-diethyl-ethylenediamine

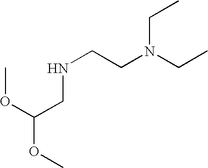

A solution of N,N-diethyl-ethylenediamine (150 g) in methanol (500 mL) was treated dropwise rapidly with glyoxal dimethylacetal (60 wt % soln. in water, 225 g) at 10-15° C. After the addition was complete the solution was warmed to 15° C., then to 22° C. and left at this temperature for 16 hr. The reaction mixture was treated with 5% palladium on carbon (Johnson-Matthey type 38H paste, 15 g) and hydrogenated at 6 bar until reaction was complete as judged by GC/MS. The catalyst was removed by filtration and the filtrate evaporated to dryness (toluene azeotrope, 2.5 L), affording 196.2 g of the sub-titled compound.

¹H NMR (300 MHz, CDCl₃): δ 4.48 (t, 1H), 3.39 (s, 6H), 2.75 (d, 2H), 2.69 (t, 2H), 2.57-2.48 (m, 6H), 1.01 (ts, 6H).

### d) 3-[2-(3-Chlorophenyl)ethoxy]-N-[2-(diethylamino)ethyl]-N-(2,2-dimethoxyethyl)propanamide

Oxalyl chloride (9.50 mL) was added dropwise over 45 minutes to a solution of 3-[2-(3-chlorophenyl)ethoxy]propanoic acid (22.50 g) (step b) in dichloromethane (120 ml) and DMF (0.5 mL) at 0° C. The reaction mixture was stirred for a further 3 hours. The mixture was subsequently concentrated, redissolved in DCM (30 ml) and added dropwise over 1 hour at 0° C. to a solution of N′-(2,2-dimethoxyethyl)-N,N-diethylethylenediamine (20.20 g) and isopropyldiethylamine (34.43 mL) in DCM (200 ml). The resulting mixture was stirred at room temperature for 3 hours, washed with aqueous saturated sodium bicarbonate solution (5×1 L), water (1.5 L) and dried over sodium sulphate and concentrated to give 39.50 g of the sub-titled compound.

m/e 415 (M+H⁺, 100%)

¹H NMR (300 MHz, CDCl₃): δ 7.26-7.07 (m, 4H), 4.51-4.38 (2×t, 1H), 3.79-3.62 (m, 4H), 3.48-3.32 (m, 100H), 2.86-2.81 (t, 2H), 2.66-2.47 (m, 8H), 1.05-0.098 (ts, 6H).

### e) 3-[2-(3-chlorophenyl)ethoxy]-N-[2-(diethylamino)ethyl]-N-(2-oxoethyl)propanamide

3-[2-(3-chlorophenyl)ethoxy]-N-[2-(diethylamino)ethyl]-N-(2,2-dimethoxyethyl)propanamide (step d) (0.5 g) was dissolved in DCM (5 ml), and the solution cooled in an ice bath. Trifluoroacetic acid (5 ml) was added in portions over 15 minutes. The mixture was allowed to stir for 2 hours. The solvent was removed in vacuo and the residue re-dissolved in DCM, then cautiously poured into saturated sodium bicarbonate solution. The organic layer was separated and the aqueous layer further extracted with DCM (×2). The organic layers were combined, dried over sodium sulphate, filtered and evaporated to give the sub-titled compound (511 mg). The material was used crude in the next step.

m/e 369 (M+H⁺)

### f) 3-[2-(3-chlorophenyl)ethoxy]-N-[2-(diethylamino)ethyl]-N-(2-{[(2R)-2-hydroxy-2-(4-hydroxy-2-oxo-2,3-dihydro-1,3-benzothiazol-7-yl)ethyl]amino}ethyl)propanamide

7-(2-Aminoethyl)-4-hydroxy-1,3-benzothiazol-2(3H)-one hydrochloride (100 mg) was dissolved in methanol (5 ml) and 3-[2-(3-chlorophenyl)ethoxy]-N-[2-(diethylamino)ethyl]-N-(2-oxoethyl)propanamide (step e) (141 mg) added, followed by acetic acid (33 ul). The mixture was stirred for 1 hour. Sodium cyanoborohydride (36 mg) was added and the mixture stirred overnight. 0.7N Ammonia in methanol was added until the reaction mixture was basic and the solvent was removed in vacuo. The residue was partitioned between ethyl acetate and water, the layers separated and the aqueous further extracted with ethyl acetate (×3). The combined organic layers were dried over sodium sulfate, filtered and evaporated to give a yellow film (130 mg). This was purified using prep HPLC (ACE column; 0.2% TFA, acetonitrile) to give a clear film. The residue was re-dissolved in acetonitrile and 0.25 ml of 4N HCl in dioxane added. The solvent was removed in vacuo, and the residue taken up in ether and stirred over the weekend. Ether decanted off to give the title compound (13 mg).

m/e 579 (M+H⁺)

¹H NMR (299.947 MHz, DMSO) δ 1.25 (t, 6H), 2.64 (t, 2H), 2.81 (t, 2H), 3.03-3.15 (m, 10H), 3.57-3.70 (m, 8H), 4.98 (t, 1H), 6.77 (d, 1H), 6.94 (d, 1H), 7.17-7.31 (m, 4H), 9.80 (s, 1H), 11.34 (s, 1H)

### Biological Assay

### Experimental Procedures

### Cell Preparation

H292 cells were grown in RPMI (Roswell Park Memorial Institute) medium containing, 10% (v/v) FBS (foetal bovine serum) and 2 mM L-glutamine. Cells were grown in 225 cm2 flasks containing 25 mL media in a humidified incubator at 37° C., 5% CO₂. Cells were harvested from the flask and passaged at a 1 in 10 dilution once per week.

### Experimental Method

The media from flasks containing H292 cells was removed, rinsed with 10 mL PBS (phosphate buffered saline) and replaced with 10 mL Accutase™ cell detachment solution. Flasks were incubated for 15 minutes in a humidified incubator at 37° C., 5% CO₂. The cell suspension was counted and the cells re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed, washed twice with 100 μL assay buffer and replaced with 50 μL assay buffer. Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds (made up as ×4 concentrated stocks in assay buffer containing 4% (v/v) dimethylsulphoxide) were added and the cells were incubated for 10 minutes at room temperature. Final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using the AlphaScreen™ methodology. The frozen cell plate was thawed for 20 minutes on a plate shaker then 10 μL of the cell lysate was transferred to a 96-well white plate. 40 μL of mixed AlphaScreen™ detection beads (containing equal volumes of donor beads (pre-incubated with biotinylated cAMP in the dark for 30 minutes) and acceptor beads), was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations (made up in lysis buffer in a 96-well tissue-culture-treated plate and frozen/thawed alongside the test samples) and detected using the same protocol. Concentration response curves for agonists were constructed to determine both the pEC₅₀ and Intrinsic Activity. Intrinsic Activity was expressed as a fraction relative to the maximum activity determined for formoterol in each experiment. The results obtained for a representative selection of the compounds of the Examples are shown in Table 1 below.

TABLE 1

| Compound of | pEC₅₀ | Intrinsic Activity |
|---|---|---|
| Example 1 | 7.7 | 0.7 |
| Example 4 | 8.9 | 1.1 |
| Example 5 | 9.5 | 1.0 |
| Example 7 | 8.6 | 1.1 |

Alternative Adrenergic (β2 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds were added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. Concentration response curves for agonists were constructed and data was fitted to a four parameter logistic equation to determine both the pEC₅₀ and Intrinsic Activity. Intrinsic Activity was expressed as a fraction relative to the maximum activity determined for formoterol in each experiment. Results for compounds of the invention are to be found in Table 2.

### Selectivity Assays

### Adrenergic α1D

### Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [³H]-prazosin (0.3 nM final concentration) and 10 μL of test compound (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Results are shown in Table 2 below.

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 nM final concentration) and 10 μL of test compound (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-odocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-odocyanopindolol binding). Results are shown in Table 2 below.

### Dopamine D2

### Membrane Preparation

Membranes containing recombinant human Dopamine Subtype D2s receptors were obtained from Perkin Elmer. These were diluted in Assay Buffer (50 mM HEPES, 1 nM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of test compound (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding).

The results obtained for a representative selection of the compounds of the Examples are shown in Table 2 below.

### Onset Assay

Dunkin-Hartley guinea-pigs (between 200 g and 300 g on delivery) were supplied by a designated breeding establishment. The guinea-pigs were killed by cervical dislocation and the trachea removed. The adherent connective tissue was removed and each trachea cut into four rings. The tissue rings were then attached to an isometric transducer. The tissues were washed and a force of 1 g was applied to each ring. In all experiments a paired curve design was used. A priming dose of 1 μM methacholine was applied to the tissues. The tissues were then washed (three times, one minute between washes), the resting tension of 1 g was reapplied and the tissues were allowed to rest for 1 hour to equilibrate. Tissues were then contracted with 1 μM methacholine and once a steady response was obtained a cumulative concentration response curve to isoprenaline (10⁻⁹ M-10⁻⁵ M) was constructed. The tissues were then washed (three times, one minute between washes) and left to rest for an hour. At the end of the resting period the tissues were contracted with 1 μM methacholine and a p[A]₅₀ concentration of test compound added. Once the tissue had reached maximum relaxation, a 30×p[A]₅₀ concentration of test compound was added. Once the tissue response had reached a plateau, 10 μM sotalol was added to the bath to confirm that the relaxation was β₂ mediated

Data were collected using the ADInstruments chart4forwindows software, which measured the maximum tension generated at each concentration of agonist.

For each concentration of the isoprenaline cumulative concentration curve, the response was calculated as % relaxation of the methacholine-induced contraction. A curve was plotted of log₁₀ [agonist] (M) versus percentage inhibition of the methacholine-induced contraction. These data were then fitted to a non-linear regression curve fit. For each experiment, E/[A] curve data were fitted using a 4-parameter logistic function of the form:

$$E = β + \frac{( β - α ) \cdot {[ A ]}^{m}}{{[ A ]}^{m} + {[ A ]}_{50}^{m}}$$

E and [A] are the pharmacological effect (% relaxation) and concentration of the agonist respectively; α, β, [A]₅₀ and m are the asymptote, baseline, location and slope parameters, respectively. The p[A]₅₀ and IA of each isoprenaline curve was determined from this fit, to determine if the tissue was viable for generating an onset time for the test compounds.

For each p[A]₅₀ concentration of the test compound, the response was calculated as % relaxation of the methacholine-induced contraction. The results were plotted % relaxation against time and the time taken to reach a 90% relaxation value was calculated and recorded.

The addition of a 30×p[A]₅₀ concentration enabled determination of the maximum compound effect within the individual tissue. Hence, the % of the maximum compound effect at the p[A]₅₀ concentration was calculated and recorded.

### Pharmacokinetics in the Rat

A dose solution of the test compound was prepared using a suitable dose vehicle. The concentration of the compound in the dose solution was assayed by diluting an aliquot to a nominal concentration of 50 μg·ml⁻¹ and calibrating against duplicate injections of a standard solution and a QC standard at this concentration. Compounds were administered intravenously as a bolus into a caudal vein to groups of three 250-350 g rats (approximately 1 ml·kg⁻¹). For the oral dose, a separate group of 2 or 3 animals were dosed by oral gavage (3 ml·kg⁻¹). Delivered doses were estimated by weight loss. Food was not usually withdrawn from animals prior to dosing, although this effect was investigated if necessary.

Blood samples (0.25 ml) were taken into 1 ml syringes from the caudal vein, transferred to EDTA tubes and plasma was prepared by centrifugation (5 min at 13000 rpm) soon after sample collection, before storage at −20° C. Typical sampling times were 2, 4, 8, 15, 30, 60, 120, 180, 240, 300 (min) or until the terminal t1/2 was accurately described.

The concentration of the analyte(s) were determined in plasma by quantitative mass spectrometry. Standard and quality control stock solutions were prepared at a concentration 1 mg/ml in methanol. A range of standard and QC stocks produced by serial dilution were added to control rat plasma (50 μl). The range of concentrations covered the range of levels of analyte present in the rat samples. Standards, QCs and samples underwent liquid extraction using 50 μl of organic solvent and 100 μl of organic solvent containing an internal standard, chosen to closely resemble the analyte. The samples were then mixed by repeated inversion, stored at −20° C. for at least 1 h, and centrifuged at 3500 rpm in a centrifuge for 20 minutes. Aliquots (120 μl) of each sample were transferred for analysis using LC-MSMS. Standard and quality control samples covering the range of concentrations found in the test samples were within 25% of the nominal concentration.

Pharmacokinetic data analysis was achieved using WinNonlin. A standard non-compartmental analysis was used to estimate the parameters such as Tmax, Cmax, Lambda_z t1/2_Lambda_z, AUCall, AUCINF (observed), Cl (observed), Vss (observed).

TABLE 2

| | β2 | β2 Int | α1 bind | β1 bind | D2 bind |
|---|---|---|---|---|---|
| Example | pEC50 | Act | pIC50 | pIC50 | pIC50 |
| 2 | 9 | 0.9 | 5.6 | 8.2 | <5.0 |
| 4 | 8.9 | 0.9 | | 8.5 | |
| 5 | 9.7 | 1 | | 7.9 | |
| 6 | 8.7 | 0.9 | 5.6 | 6.2 | 5.6 |
| 7 | 8.4 | 1 | 5.3 | | 5.3 |
| 9 | 8.4 | 0.9 | 5.7 | 5.8 | <5.0 |

1. A compound of formula (I): 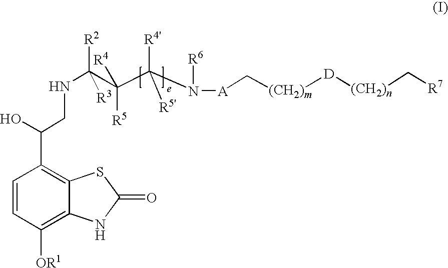
wherein
R¹ represents hydrogen or benzyl;
each of R², R³, R⁴, R⁵, R^{4′} and R^{5′} independently represents hydrogen or C₁-C₆ alkyl;
e is 0 or 1;
A represents CH₂, C(O) or S(O)₂;
D represents oxygen, sulphur or NR⁸;
m is an integer from 0 to 3;
n is an integer from 0 to 3;
R⁶ represents a group —(X)ₚ—Y-(Z)_{q}-R¹⁰;
X and Z each independently represent a C₁-C₆ alkylene group optionally substituted by halogen, trifluoromethyl, amino (NH₂), (di)-C₁-C₆ alkylamino, (di)-C₁-C₆ alkylaminocarbonyl, C₁-C₆ alkylcarbonylamino, sulphonamido (—SO₂NH₂) or (di)-C₁-C₆ alkylaminosulphonyl;
p and q each independently represent 0 or 1;
Y represents a bond, oxygen, sulphur, CH₂, C(O) or NR⁹;
R⁸ represents hydrogen or C₁-C₆ alkyl;
R⁹ represents hydrogen or C₁-C₆ alkyl;
R¹⁰ represents hydrogen, or a saturated or unsaturated 3- to 10-membered ring system comprising none, one or more heteroatoms selected from nitrogen, oxygen and sulphur, the ring system being optionally substituted by halogen, trifluoromethyl, cyano, carboxyl, hydroxyl, nitro, —S(O)ᵣR¹⁵, —NR¹⁶S(O)ₛR¹⁷, —C(O)NR¹⁸R¹⁹, —NHC(O)R²⁰, C₁-C₆ alkyl, C₁-C₆ alkoxy, C₁-C₆ alkylcarbonyl, C₁-C₆ alkoxycarbonyl or a saturated or unsaturated 4- to 7-membered monocyclic ring system comprising none, one or more ring heteroatoms selected from nitrogen, oxygen and sulphur, the monocyclic ring system itself being optionally substituted by halogen, trifluoromethyl, hydroxyl, —NR²¹S(O)ₜR²², —NHC(O)R²³ or C₁-C₆ alkoxy;
R¹⁶, R¹⁸, R¹⁹, R²⁰, R²¹ and R²³ each independently represent hydrogen or C₁-C₆ alkyl; R¹⁵, R¹⁷ and R²² are, independently, C₁-C₆ alkyl;
r, s and t each independently represent 0, 1 or 2;
R⁷ represents a 5- to 14-membered aromatic or heteroaromatic ring system optionally substituted by halogen, trifluoromethyl, hydroxyl, carboxyl, C₁-C₆ alkyl (optionally substituted by —NR²⁴R²⁵), C₁-C₆ alkoxy (optionally substituted by —NR²⁶R²⁷), C₁-C₆ alkoxycarbonyl, —NR²⁸R²⁹, C₁-C₆ alkylcarbonylamino, C₁-C₆ alkylsulphonylamino, phenylsulphonylamino, —C(O)NHR³⁰, —SO₂NHR³³, C₀-C₆ alkyl-R³⁴, or a phenyl or 5- to 6-membered heteroaromatic ring (each of which is optionally substituted by halogen, trifluoromethyl, hydroxyl, C₁-C₆ alkyl, C₁-C₆ alkoxy or —NR³⁵R³⁶);
R²⁴, R²⁵, R²⁶, R²⁷, R²⁸ and R²⁹ each independently represent hydrogen or C₁-C₆ alkyl;
R³⁰ represents hydrogen, C₁-C₆ alkyl, phenyl-C₀-C₆ alkyl or C₂-C₆ alkylene-NR³¹R³²;
either R³¹ and R³² each independently represent hydrogen or C₁-C₆ alkyl, or R³¹ and R³² together with the nitrogen atom to which they are attached form a 4- to 6-membered saturated heterocyclic ring optionally comprising a further ring heteroatom selected from nitrogen and oxygen;
R³³ represents hydrogen, C₁-C₆ alkyl, phenyl-C₀-C₆ alkyl or C₂-C₆ alkylene-NR³⁷R³⁸;
R³⁴ represents a saturated, 5- or 6-membered nitrogen-containing ring;
R³⁵ and R³⁶ each independently represent hydrogen or C₁-C₆ alkyl; and
either R³⁷ and R³⁸ each independently represent hydrogen or C₁-C₆ alkyl, or R³⁷ and R³⁸ together with the nitrogen atom to which they are attached form a 4- to 6-membered saturated heterocyclic ring optionally comprising a further ring heteroatom selected from nitrogen and oxygen;
or a pharmaceutically acceptable salt thereof.

2. A compound as claimed in claim 1 wherein R¹ is hydrogen.

3. A compound as claimed in claim 1 wherein R², R³, R⁴, R⁵, R^{4′} and R^{5′} are all hydrogen.

4. A compound according to claim 1, wherein A represents C(O) or CH₂.

5. A compound as claimed in claim 1 wherein m and n are both 1.

6. A compound as claimed in claim 1 wherein D represents oxygen.

7. A compound as claimed in claim 1 wherein p and q each independently represent 0 or 1, X and Z each independently represent an unsubstituted C₁-C₆ alkylene group and Y represents a bond, CH₂ or NR⁹.

8. A compound as claimed in claim 1 wherein R¹⁰ represents hydrogen, or a saturated or unsaturated 5- or 6-membered ring system optionally comprising one or two ring heteroatoms independently selected from nitrogen and oxygen, the ring system being optionally substituted by halogen, trifluoromethyl, carboxyl, hydroxyl, —S(O)ᵣR¹⁵, —NR¹⁶S(O)ₛR¹⁷, —C(O)NR¹⁸R¹⁹, —NHC(O)R²⁰, C₁-C₄ alkyl, C₁-C₄ alkoxy, C₁-C₄ alkylcarbonyl or C₁-C₄ alkoxycarbonyl.

9. A compound as claimed in claim 1 wherein R⁶ is hydrogen, phenyl or (CH₂)ₖR^{10a}; wherein k is 1, 2, 3 or 4; R^{10a} is hydrogen, phenyl or NR^{9a}R^{9b}; and R^{9a} and R^{9b} are, independently, C₁₋₄ alkyl.

10. A compound as claimed in claim 1 wherein R⁷ represents a 6- to 10-membered aromatic ring system optionally substituted by none, one or two substituents independently selected from halogen, trifluoromethyl, hydroxyl, carboxyl, C₁-C₄ alkyl (optionally substituted by none, one or two —NR²⁴R²⁵), C₁-C₄ alkoxy (optionally substituted by none, one or two —NR²⁶R²⁷), C₁-C₄ alkoxycarbonyl, —NR²⁸R²⁹, C₁-C₄ alkylcarbonylamino, C₁-C₄ alkylsulphonylamino, phenylsulphonylamino, —C(O)NHR³⁰, —SO₂NHR³³, C₀-C₄ alkyl-R³⁴, phenyl and a 5- to 6-membered heteroaromatic ring.

11. A compound according to claim 1 which is:
N-[2-[2-(4-Benzyloxy-2-oxo-3H-benzothiazol-7-yl)-2-hydroxy-ethyl]aminoethyl]-N-butyl-3-phenethyloxy-propanamide,
N-Butyl-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide,
N-[2-({(2R)-2-[4-(Benzyloxy)-2-oxo-2,3-dihydro-1,3-benzothiazol-7-yl]-2-hydroxyethyl}amino)ethyl]-3-(2-phenylethoxy)-N-(2-phenylethyl)propanamide,
N-(2-{[(2R)-2-Hydroxy-2-(4-hydroxy-2-oxo-2,3-dihydro-1,3-benzothiazol-7-yl)ethyl]amino}ethyl)-3-(2-phenylethoxy)-N-(2-phenylethyl)propanamide,
N-(2-{[(2R)-2-Hydroxy-2-(4-hydroxy-2-oxo-2,3-dihydro-1,3-benzothiazol-7-yl)ethyl]amino}ethyl)-N-methyl-3-(2-phenylethoxy)propanamide,
N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[(2R)-2-hydroxy-2-(4-hydroxy-2-oxo-2,3-dihydro-1,3-benzothiazol-7-yl)ethyl]amino}ethyl)-3-(2-phenylethoxy)propanamide,
4-Hydroxy-7-{(1R)-1-hydroxy-2-[(2-{[3-(2-phenylethoxy)propyl]amino}-ethyl)amino]ethyl}-1,3-benzothiazol-2(3H)-one, or,
3-[2-(3-Chlorophenyl)ethoxy]-N-[2-(diethylamino)ethyl]-N-(2-{[(2R)-2-hydroxy-2-(4-hydroxy-2-oxo-2,3-dihydro-1,3-benzothiazol-7-yl)ethyl]amino}ethyl)propanamide,
or a pharmaceutically acceptable salt thereof.

12. A process for the preparation of a compound of formula (I) or a pharmaceutically acceptable salt thereof as defined in claim 1 which comprises:
(a) reacting a compound of formula (II) 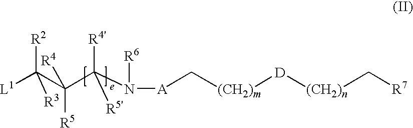
wherein L¹ represents a leaving group and e, R², R³, R⁴, R⁵, R^{4′}, R^{5′}, R⁶, R⁷, A, D, m and n are as defined in formula (I), with a compound of formula (III) or a suitable salt thereof. 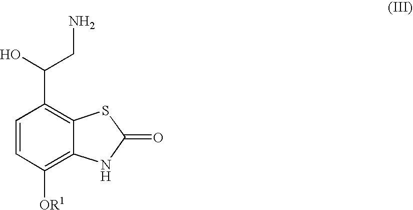
wherein R¹ is as defined in formula (I), in the presence of a base; or
(b) when R² and R³ each represent hydrogen, reacting a compound of formula (IV) 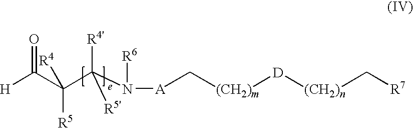
wherein e, R⁴, R⁵, R^{4′}, R^{5′}, R⁶, R⁷, A, D, m and n are as defined in formula (I), with a compound of formula (III) or a suitable salt thereof as defined in (a) above in the presence of a suitable reducing agent; or
(c) when R² and R³ each represent hydrogen, contacting a compound of formula (V) 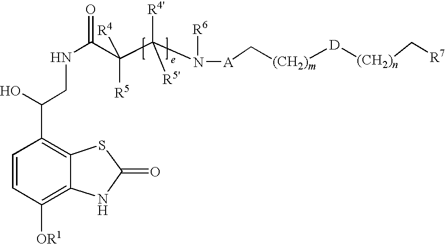
wherein e, R¹, R⁴, R⁵, R^{4′}, R^{5′}, R⁶, R⁷, A, D, m and n are as defined in formula (I) with a suitable reducing agent;
and optionally after (a), (b) or (c) carrying out one or more of the following:
converting the compound obtained to a further compound of formula (I)
forming a pharmaceutically acceptable salt of the compound.

13. A compound of formula (III): 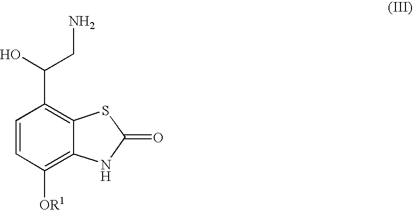
wherein R¹ is as defined in claim 1.

14. A compound of formula:

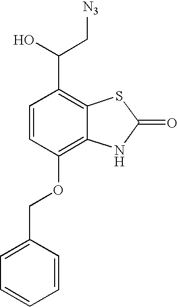

15. A pharmaceutical composition comprising a compound of formula (I) or a pharmaceutically acceptable salt thereof as claimed in claim 1 in association with a pharmaceutically acceptable adjuvant, diluent or carrier.

16. A process for the preparation of a pharmaceutical composition as which comprises mixing a compound of formula (I) or a pharmaceutically acceptable salt thereof as claimed in claim 1 with a pharmaceutically acceptable adjuvant, diluent or carrier.

17-19. (canceled)

20. A method of treating, or reducing the risk of, a disease or condition in which modulation of β2 adrenoreceptor activity is beneficial which comprises administering to a patient in need thereof a therapeutically effective amount of a compound of formula (I) or a pharmaceutically acceptable salt thereof as claimed in claim 1.

21. A method of treating, or reducing the risk of, an inflammatory disease or condition which comprises administering to a patient in need thereof a therapeutically effective amount of a compound of formula (I) or a pharmaceutically acceptable salt thereof as claimed in claim 1.

22. A method according to claim 20, wherein the disease or condition is adult respiratory distress syndrome (ARDS), pulmonary emphysema, bronchitis, bronchiectasis, chronic obstructive pulmonary disease (COPD), asthma or rhinitis.

23. A combination comprising a compound of formula (I) and one or more active agents selected from the list comprising:
a PDE4 inhibitor including an inhibitor of the isoform PDE4D;
a glucocorticoid receptor agonist;
a muscarinic receptor antagonist;
a modulator of chemokine receptor function; or,
an inhibitor of p38 kinase function.

24. A method according to claim 21, wherein the disease or condition is adult respiratory distress syndrome (ARDS), pulmonary emphysema, bronchitis, bronchiectasis, chronic obstructive pulmonary disease (COPD), asthma or rhinitis.
